(19) United States
(12) Patent Application Publication
(10) Pub. No.: US 2026/0192094 A1
(43) Pub. Date: Jul. 9, 2026

(54) METHODS OF APPLYING DRUG DELIVERY COATINGS TO MEDICAL BALLOONS AND RELATED DEVICES FOR CREATING DRUG COATED BALLOONS

(71) Applicant: Wake Forest University Health Sciences, Winston-Salem, NC (US)

(72) Inventor: Saami Yazdani, Winston-Salem, NC (US)

(21) Appl. No.: 19/555,123

(22) Filed: Mar. 3, 2026

Related U.S. Application Data
(63) Continuation-in-part of application No. PCT/US2024/040017, filed on Jul. 29, 2024.
(60) Provisional application No. 63/535,336, filed on Aug. 30, 2023.

(51) Int. Cl. A61M 25/10

(52) U.S. Cl. CPC A61M 25/1029; A61M 25/104; A61M 2025/1031; A61M 2025/105; A61M 2025/1081

(57) ABSTRACT

Systems and methods for applying drug delivery coatings to one or more uncoated balloon catheters for a medical procedure include a tube comprising an inner surface surrounding a lumen. The inner surface comprises at least one therapeutic agent. An uncoated balloon of a balloon catheter is inserted into the lumen of the tube and expanded to contact the inner surface whereby the at least one therapeutic agent is applied to the balloon to thereby provide/create a drug coated balloon for the medical procedure.

### RELATED APPLICATIONS

This patent application is a continuation-in-part of and claims priority to PCT Application Number PCT/US2024/040017, filed July 29, 2024, which claims priority to U.S. Provisional Application Serial Number 63/535,336, filed August 30, 2023, the contents of which are hereby incorporated by reference as if recited in full herein.

### FIELD OF THE INVENTION

The present invention relates to surgical devices with intrabody (e.g., intravascular) balloon catheters configured to deliver a therapy.

### BACKGROUND

Cardiovascular disease is the leading cause of death globally. Atherosclerosis, the buildup of fats, cholesterol and other substances within the arterial wall, is a vascular proliferative disease mostly commonly observed in older adults, blocking blood flow to your heart, brain, arms, legs and other vital organs. Percutaneous intervention, i.e., balloon angioplasty and stenting, is the preferred choice to treat these flow-limiting arterial blockages due to reduced morbidity, mortality, and in-hospital stays as compared to bypass surgery ^{1, 2}. See, e.g., Norgren L, Hiatt WR, Dormandy JA, Nehler MR, Harris KA, Fowkes FG, Bell K, Caporusso J, Durand-Zaleski I, Komori K, et al. Inter-Society Consensus for the Management of Peripheral Arterial Disease (TASC II). European journal of vascular and endovascular surgery : the official journal of the European Society for Vascular Surgery. 2007;33 Suppl 1:S1-75. doi: 10.1016/j.ejvs.2006.09.024; and Tsetis D, Belli AM. Guidelines for stenting in infrainguinal arterial disease. Cardiovascular and interventional radiology. 2004;27:198-203. doi: 10.1007/s00270-004-0029-1.

However, 50-85% of patients develop significant restenosis and 16-65% develop occlusion within two years of treatment ^{3,4}. See, e.g., Schillinger M, Sabeti S, Dick P, Amighi J, Mlekusch W, Schlager O, Loewe C, Cejna M, Lammer J, Minar E. Sustained benefit at 2 years of primary femoropopliteal stenting compared with balloon angioplasty with optional stenting. Circulation. 2007;115:2745-2749. doi: 10.1161/circulationaha.107.688341; and Tosaka A, Soga Y, Iida O, Ishihara T, Hirano K, Suzuki K, Yokoi H, Nanto S, Nobuyoshi M. Classification and clinical impact of restenosis after femoropopliteal stenting. J Am Coll Cardiol. 2012;59:16-23. doi: 10.1016/j.jacc.2011.09.036.

The use of anti-proliferative drugs in combination with bare metal stents, i.e., drug-eluting stents (DES), was a major breakthrough in treating coronary artery disease ^{5,6}. See, Stone GW, Ellis SG, Cox DA, Hermiller J, O'Shaughnessy C, Mann JT, Turco M, Caputo R, Bergin P, Greenberg J, et al. A polymer-based, paclitaxel-eluting stent in patients with coronary artery disease. N Engl J Med. 2004;350:221-231. doi: 10.1056/NEJMoa032441; and Morice MC, Serruys PW, Sousa JE, Fajadet J, Ban Hayashi E, Perin M, Colombo A, Schuler G, Barragan P, Guagliumi G, et al. A randomized comparison of a sirolimus-eluting stent with a standard stent for coronary revascularization. N Engl J Med. 2002;346:1773-1780. doi: 10.1056/NEJMoa012843. The contents of these documents are hereby incorporated by reference as if recited in full herein.

However, DES have shown subpar outcomes in the treatment of peripheral arterial disease mainly because stents are subjected to biomechanical stress and severe artery deformation (twisting, bending, and shortening), leading to high fracture rates (up to 68%) and restenosis⁷. See, Scheinert D, Scheinert S, Sax J, Piorkowski C, Braunlich S, Ulrich M, Biamino G, Schmidt A. Prevalence and clinical impact of stent fractures after femoropopliteal stenting. J Am Coll Cardiol. 2005;45:312-315. doi: 10.1016/j.jacc.2004.11.026. Drugs eluted from stents are also constrained to the platform design and may only deliver drugs to about 20% or less of the arterial surface⁸. See, Hwang CW, Wu D, Edelman ER. Physiological transport forces govern drug distribution for stent-based delivery. Circulation. 2001;104:600-605.

Additionally, following complete elution of drugs from a DES, a persistent metal scaffold and polymeric coatings can serve as a potential source for inflammation and neoatherosclerosis.

Drug coated balloons (DCBs) have emerged as a promising new therapeutic approach to treat occlusive arterial disease and allow interventionalists the ability to ‘leave nothing behind,’ preserving future treatment options. DCBs were developed by coating an uncoated angioplasty balloon with the anti-proliferative drug paclitaxel and showed their clinical efficacy in multicenter, randomized controlled trials of patients with symptomatic femoropopliteal artery disease comparing DCBs to standard balloon angioplasty. The first DCB to achieve FDA approval for de novo or restenotic femoropopliteal artery lesions in the United States was Lutonix (BARD, Murray Hill, NJ) in October 2014. The second DCB to achieve FDA approval was IN.PACT Admiral (Medtronic, Minneapolis, MN) in December 2014. Shortly after DCBs become commercially available, the Centers for Medicare and Medicaid Services (CMS) initiated an add-on-payment for a transitional pass-through (TPT) and new technology add-on payments in 2015. Subsequently, however, CMS made the determination to end the add-on payments and instead package DCB device costs into the currently available payment for uncoated balloon angioplasty⁹. See, e.g., Shishehbor MH, Jaff MR, Beckman JA, Misra S, Schneider PA, Lookstein R, Kashyap VS, Aronow HD, Jones WS, White CJ. Public Health Impact of the Centers for Medicare and Medicaid Services Decision on Pass-Through Add-On Payments for Drug-Coated Balloons: A Call to Action. JACC Cardiovascular interventions. 2018;11:496-499. doi: 10.1016/j.jcin.2018.01.233. The content of this document is hereby incorporated by reference as if recited in full herein.

Considering that reimbursement for revascularization with transluminal balloon angioplasty can be as low as $3,000 and the costs of DCBs range anywhere between $1,000 to $2,000 each, the economics of using DCBs has been severely hampered, therefore limiting the use of this favorable technology which has shown to improve clinical outcomes and reduce repeat vascularization procedures.

Thus, while results from numerous clinical outcomes has shown improvement in patency, in particular the periphery and hemodialysis access, widespread adoption has been hampered due to costs. Thus, there is a need for cost-effective methods and systems to provide DCBs.

### SUMMARY

Embodiments of the present invention provide a tube having a wall with an inner surface surrounding a lumen. The inner surface has with at least one therapeutic agent deposited thereon. The inner surface of the tube is configured to contact an outer surface of the expandable balloon, when the expandable balloon is in an expanded state, to apply the at least one therapeutic agent to the outer surface of the balloon to thereby provide a drug coated balloon for a medical procedure.

The tube or multiple tubes can be provided in a medical kit for onsite use by a clinician. A single tube can be used to apply the at least one therapeutic agent (which can be the same or different from each other) to a respective balloon during a respective medical procedure. The balloon can then be re-coated using another single tube of the multiple tubes, avoiding the need for multiple different DCBs for the respective medical procedure.

A respective single tube can be configured to coat different size length and/or outer diameter balloons of a balloon catheter.

Embodiments of the invention are directed to systems for providing a drug coated balloon of a balloon catheter for a medical procedure. The systems include a tube having an inner surface surrounding a lumen. The inner surface has at least one therapeutic agent. The systems also include a catheter that includes a balloon. The balloon is sized and configured to releasably reside in the lumen and the balloon is expandable in the lumen of the tube to contact the inner surface whereby the at least one therapeutic agent is applied to the balloon to thereby provide a drug coated balloon for the medical procedure.

The balloon can be precoated with an excipient that resides between the inner surface and the at least one therapeutic agent.

The excipient can include or be a contrast agent.

The at least one therapeutic agent can inhibit and/or block restenosis of an artery, stented artery, vein, stented vein, and/or urethra.

The tube can have an elongate body with opposing first and second end portions and at least one of the first and second end portions can include a valved luer fitting.

The tube can be provided in sterile packaging prior to the medical procedure

The tube can have an elastic body surrounding the lumen and providing the inner surface.

The tube can have an inelastic and/or rigid body surrounding the lumen and providing the inner surface.

The tube can be provided as a component of a kit for angioplasty balloon therapy.

In a non-expanded state, the tube can have an inner diameter in a range of 1.5 mm to 15 mm.

The tube can be a first tube. The system can further include a second tube having an inner surface surrounding a lumen. The inner surface can have at least one therapeutic agent. The balloon is expandable in the lumen of the second tube to contact the inner surface whereby the at least one therapeutic agent can be applied to the balloon to thereby provide another coating to the balloon for the medical procedure thereby providing a multi-use drug coated balloon that serially delivers at least one therapeutic agent applied by the first and second tubes to different target sites or to a single target site twice during a single medical procedure.

Naiive balloon catheters can be used with the first tube and the second tube to deliver at least one therapeutic agent applied by the first tube and/or the second tube to a single target site at least twice or to different target sites during a single medical procedure instead of reusing a respective balloon catheter.

The tube can be sufficiently compliant to correspond to compliancy of a blood vessel. The balloon can expand from a deflated configuration to conformably reside against the inner surface of the tube for coating the balloon.

Other aspects of the present invention are directed to methods for coating a balloon to provide a drug coated balloon for a medical procedure. The methods include providing a tube with an inner surface surrounding a lumen and coating the at least one therapeutic agent onto the inner surface.

The method can include sterilizing the tube before and/or after the inserting step.

The tube can have an inner diameter in a range of 1.5-15 mm.

The tube can have a length that is in a range of 10-350 mm.

The tube can have a wall that provides the inner surface.

The wall can be expandable. The wall can be non-expandable.

The tube can be formed of an elastic material and/or can have at least one valved luer fitting.

The tube can be formed of an inelastic material and/or can have at least one valved luer fitting.

The coating can be carried out by micro-pipetting, spraying or introducing a liquid solution with the at least one therapeutic agent into the lumen.

If introducing liquid solution presents excess liquid, then the method of coating can include removing excess liquid solution from the lumen, then drying the at least one therapeutic agent on the inner surface to provide the coating.

The tube can have an elongate body with opposing first and second end portions and at least one of the first and second end portions can have a valved luer connector sized and be configured to releasably receive a portion of a catheter comprising a balloon.

The at least one therapeutic agent can inhibit or blocks restenosis of an artery, stented artery, vein, stented vein or urethra.

The coating can be carried out to cover an entire circumferential region of the inner surface for at least a major portion of a length of the lumen.

Yet other aspects of the present invention are directed to methods for providing a drug coated balloon. The methods include: providing a balloon catheter; providing a tube with an inner surface having at least one therapeutic agent; inserting a balloon of the balloon catheter into a lumen of the tube and expanding the balloon therein so that an outer surface of the expanded balloon contacts the inner surface of the tube; transferring at least some of the at least one therapeutic agent onto the outer surface of the expanded balloon to provide a drug coated balloon; and (at least partially) deflating the drug coated balloon; then removing the drug coated balloon from the tube.

The method can include performing a medical procedure whereby the at least one therapeutic agent is delivered to a target intrabody site of a patient.

The medical procedure can be a balloon angioplasty.

The inserting, transferring, deflating and performing actions can be carried out in a surgical room.

The tube can be a first tube. The method can further include: providing a second tube with an inner surface having at least one therapeutic agent; inserting the balloon of the balloon catheter or inserting a balloon of a new balloon catheter into a lumen of the second tube and expanding the balloon therein so that an outer surface of the expanded balloon contacts the inner surface of the second tube; transferring at least some of the at least one therapeutic agent onto the outer surface of the expanded balloon to recoat the balloon; deflating the recoated balloon and removing the recoated balloon from the second tube; and serially reusing the balloon catheter to treat a target site twice or to treat two or more different target sites with the same balloon and the at least one therapeutic agent transferred from the inner surface of the first tube then the at least one therapeutic agent transferred from the inner surface of the second tube.

The at least one therapeutic agent can inhibit or block restenosis of an artery, stented artery, vein, stented vein and/or urethra.

The method can include pre-coating the balloon with a contrast agent or other excipient before the inserting step.

The method can further include holding the expanded balloon in the lumen for a defined time, optionally in a range of 30 seconds to 10 minutes, for the transfer of at least some of the at least one therapeutic agent.

Yet other aspects of the present invention are directed to a tube assembly providing a drug coating system for a catheter having a balloon. The tube assembly includes an elongate tube having a wall surrounding a lumen. An inner surface of the wall with a coating that includes at least one therapeutic agent. The tube assembly also includes a luer connector on a first end portion of the elongate tube. The luer connector and tube are sized and configured to slidably receive the balloon of the catheter when the balloon is in a deflated state.

The tube assembly can be provided in a medical grade sterile package.

The luer connector can be a valved luer connector.

The at least one therapeutic agent can block or inhibit restenosis.

The tube assembly can also include a second luer fitting on a second end portion of the tube and an end cap releasably attached to the second luer fitting.

The tube can have an inner diameter in a range of 1.5 mm to 15 mm.

The tube can be an elastic tube.

The tube can be an inelastic tube.

The coating can provide the at least one therapeutic agent to an artery in a range of 0.01 to 100 ng/mg or greater.

The coating on the balloon can provide the at least one therapeutic agent in an amount in a range of 0.01 μg/mm² to 10 μg/mm² or greater.

Yet other aspects of the present invention are directed to systems for providing a drug coated balloon of a balloon catheter for a medical procedure. The systems include a tube configured to couple to an intravascular introducer sheath or provided by a longitudinally extending segment of the intravascular introducer sheath, the tube including an inner surface surrounding a lumen, wherein the inner surface comprises at least one therapeutic agent deposited thereon. The systems also include a catheter including a balloon coupled to the introducer sheath, wherein the balloon is sized and configured to slidably reside in the lumen of the tube then slidably extended into position in the patient to target anatomy through the intravascular sheath for a medical procedure, and wherein the balloon is expandable in the lumen of the tube to contact the inner surface for the defined period whereby the at least one therapeutic agent is applied to the balloon to thereby provide a drug coated balloon for the medical procedure.

The balloon can be precoated with an excipient that resides between the inner surface and the at least one therapeutic agent.

The excipient can include a contrast agent.

The at least one therapeutic agent can inhibit and/or block restenosis of an artery, stented artery, vein, stented vein or urethra.

The tube can include an elongate body with opposing first and second end portions, and wherein at least one of the first and second end portions can include a valved luer fitting.

The tube can have an elastic body surrounding the lumen and providing the inner surface.

The tube can have an inelastic and/or substantially rigid body surrounding the lumen and providing the inner surface.

The tube can be provided as a component of a kit for angioplasty balloon therapy.

Prior to use the tube can have an inner diameter in a range of 1.5 mm to 15 mm.

The tube can be sufficiently compliant to correspond to compliancy of a blood vessel, and wherein the balloon expands from a deflated configuration to conformably reside against the inner surface of the tube for coating the balloon, then is deflated again and advanced through the intravascular introducer sheath to the target anatomy.

The tube can be coupled to the intravascular sheath at a location that is external to the patient.

The tube can include a valve between a connector of the intravascular sheath and a distal end of the tube.

The tube can be provided as a longitudinally extending segment of the intravascular sheath at a location that is distal to a proximal connector of the intravascular sheath.

The systems can also include a conduit extending laterally from a proximal end portion of the intravascular sheath in fluid communication with the tube.

The systems can also include a guidewire sized and configured to slidably extend through the catheter and the intravascular sheath.

Yet other aspects of the present invention are directed to methods for providing a drug coated balloon for a medical procedure. The methods include: providing a balloon catheter; providing a tube with an inner surface including at least one therapeutic agent; providing an intravascular introducer sheath; coupling the intravascular introducer sheath to the tube; then inserting a balloon of the balloon catheter into a lumen of the tube and expanding the balloon therein so that an outer surface of the expanded balloon contacts the inner surface of the tube; transferring at least some of the at least one therapeutic agent onto the outer surface of the expanded balloon to provide a drug coated balloon; at least partially deflating the drug coated balloon; and extending the drug coated balloon through the intravascular sheath to target anatomy thereby providing an in situ drug coated balloon for a medical procedure.

The drug coated balloon can be configured to deliver a therapeutic amount of the at least one therapeutic agent to a target intrabody site of a patient.

The medical procedure can be a balloon angioplasty.

The method can include holding the expanded balloon in the lumen for a defined time, optionally in a range of 30 seconds-10 minutes, for the transfer of at least some of the at least one therapeutic agent.

Yet other aspects of the present invention are directed to methods for providing a drug coated balloon for a medical procedure. The methods include: providing a balloon catheter; providing an intravascular introducer sheath including a longitudinally extending segment with an inner wall including an inner surface including at least one therapeutic agent; coupling the intravascular introducer sheath to the tube; then inserting a balloon of the balloon catheter into a lumen of the tube and expanding the balloon therein so that an outer surface of the expanded balloon contacts the inner surface of the tube; transferring at least some of the at least one therapeutic agent onto the outer surface of the expanded balloon to provide a drug coated balloon; at least partially deflating the drug coated balloon; and extending the drug coated balloon through the intravascular sheath to target anatomy thereby providing an in situ drug coated balloon for a medical procedure.

The drug coated balloon can be configured to deliver a therapeutic amount of the at least one therapeutic agent to a target intrabody site of a patient.

The medical procedure can be a balloon angioplasty.

Yet other aspects of the present invention are directed to intravascular introducer sheath systems including an intravascular introducer sheath including a proximal end and an opposing distal end, wherein the intravascular introducer sheath includes a sheath body coupled at a proximal end to a connector, wherein the sheath body surrounds a longitudinally extending lumen bounded by an inner surface of a wall of the sheath body, and wherein a longitudinally extending segment of the inner surface of the sheath body includes at least one therapeutic agent configured to coat a balloon of a balloon catheter.

The connector can be a luer connector sized and configured to slidably receive the balloon of the catheter when the balloon is in a deflated state.

The at least one therapeutic agent can block or inhibit restenosis, optionally wherein the at least one therapeutic agent includes paclitaxel and/or sirolimus.

Further features, advantages and details of the present invention will be appreciated by those of ordinary skill in the art from a reading of the figures and the detailed description of the preferred embodiments that follow, such description being merely illustrative of the present invention.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

### BRIEF DESCRIPTION OF DRAWINGS

FIGS. 1-6 are schematic illustrations of a sequence of actions that can be carried out to provide a drug coated balloon according to embodiments of the present invention.

FIGS. 7A-7E are top views of an example tube assembly and an example sequence of actions that can be carried out according to embodiments of the present invention. FIG. 7B shows the tube assembly coupled to a syringe comprising a liquid solution of at least one therapeutic agent. FIG. 7E shows the tube assembly with a coating on the inner surface and in packaging for sterilization or post sterilization for applying the coating to a balloon for a medical procedure.

FIGS. 8A-8D show a sequence of actions that can be used to provide a drug coated balloon using the tube assembly shown in FIG. 7E. FIG. 8A shows an uncoated balloon positioned into a bowl containing contrast agent. FIG. 8B shows the uncoated balloon catheter placed within the drug-lined tube. FIG. 8C shows the uncoated balloon deployed within the drug-lined tube. FIG. 8D shows the drug coated balloon deflated and removed from the tube, ready to be positioned and deployed within a patient (e.g., an artery).

FIG. 9 is a block diagram of an example medical kit that can be provided to provide a drug coated balloon catheter according to embodiments of the present invention.

FIG. 10A is a flow chart of example actions that can be used to provide a tube for coating a balloon catheter according to embodiments of the present invention.

FIG. 10B is a flow chart of example actions that can be used to coat a balloon catheter with at least one therapeutic agent provided by a tube comprising an inner surface with at least one therapeutic agent according to embodiments of the present invention.

FIG. 11 is a schematic illustration of an intravascular introducer sheath in combination with a tube configured to coat a balloon of a balloon catheter in situ with at least one therapeutic agent according to embodiments of the present invention.

FIG. 12A is a side view of an example intravascular introducer sheath coupled to a tube with the tube configured to coat a balloon of a balloon catheter and with a guide wire extending through the tube and through the intravascular sheath prior to the balloon being slidably positioned in the tube according to embodiments of the present invention.

FIG. 12B is a side view of the example intravascular introducer sheath coupled to a tube with the tube shown in FIG. 12A, showing an uncoated balloon of a balloon catheter proximal to the tube and with a guide wire extending through the tube and through the intravascular sheath prior to the balloon being slidably positioned in the tube according to embodiments of the present invention.

FIG. 12C is a side view of the example intravascular introducer sheath coupled to a tube shown in FIG. 12B, but with the balloon in the tube to apply the coating, upon at least partial inflation of the balloon, prior to the balloon of the balloon catheter being slidably advanced through the intravascular sheath in a deflated state to target anatomy to be inflated to deliver a therapy by transferring a therapeutically effective amount of the applied coating according to embodiments of the present invention.

FIG. 13 is a schematic illustration of an intravascular introducer sheath that has an integrated tube segment configured to coat a balloon of a balloon catheter with at least one therapeutic agent according to embodiments of the present invention.

FIG. 14A is a schematic diagram of an ex vivo flow circuit system used to provide pulsatile flow conditions for a bioreactor.

FIGS. 14B and 14C are digital images of a bioreactor housing compartment of the ex vivo flow circuit system shown in FIG. 14A, with explanted pig artery and a coated balloon for evaluation of biokinetic action provided by the coated balloon according to embodiments of the present invention.

FIG. 15A is a scanning electron microscopy (SEM) image of a surface of an uncoated balloon angioplasty.

FIG. 15B is an SEM image of an uncoated balloon angioplasty balloon following transfer of a drug from the drug-lined tube.

FIGS. 15C and 15D are SEM images of a luminal surface of an artery showing drug transfer from the balloon to the treatment site of the target artery.

FIG. 16A is an SEM image of dissolved sirolimus showing phenotype characteristics.

FIGS. 16B and 16C are SEM images of a luminal surface of an artery showing drug transfer from a balloon coated with dissolved sirolimus at 6 mg/mL (FIG. 16B) and 12 mg/mL (FIG. 16C).

FIG. 16D is an SEM image of dissolved paclitaxel showing phenotype characteristics.

FIGS. 16E and 16F are SEM images of a luminal surface of an artery showing drug transfer from a balloon coated with dissolved paclitaxel at 6 mg/mL (FIG. 16E) and 12 mg/mL (FIG. 16F).

FIG. 17A is a side perspective view of an example spray coating system aligned with a tube to coat the inner surface of the tube with at least one therapeutic agent according to embodiments of the present invention.

FIG. 17B is a side perspective view of another example spray coating system aligned with a tube to coat the inner surface of the tube with at least one therapeutic agent according to embodiments of the present invention.

FIG. 18 is an SEM image of 12 mg/ml of dissolved solid paclitaxel spray coated onto silicone tubing using the spray coating system shown in FIG. 17A.

FIGS. 19A and 19B are SEM images of 12 mg/ml of dissolved solid paclitaxel with contrast as the excipient coated onto the inner surface of a silicone tube according to embodiments of the present invention.

FIG. 20 is a digital photograph of a drug lined tube that was used to coat outer surfaces of different size balloons, 5 mm, 6 mm, 7 mm (shown by SEM images on the right side of the figure).

### DETAILED DESCRIPTION

The present invention will now be described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. The abbreviation “FIG.” may be used interchangeably with “Fig.” and the word “Figure” in the specification and figures. It will be appreciated that although discussed with respect to a certain embodiment, features or operation of one embodiment can apply to others.

In the drawings, the thickness of lines, layers, features, components and/or regions may be exaggerated for clarity and broken lines (such as those shown in flow diagrams) illustrate optional features or operations, unless specified otherwise. In addition, the sequence of operations (or steps) is not limited to the order presented in the claims unless specifically indicated otherwise.

The terminology used herein is for the purpose of describing particular embodiments only and is not intended to be limiting of the invention. As used herein, the singular forms "a", "an" and "the" are intended to include the plural forms as well, unless the context clearly indicates otherwise. It will be further understood that the terms "comprises" and/or "comprising," when used in this specification, specify the presence of stated features, steps, operations, elements, and/or components, but do not preclude the presence or addition of one or more other features, steps, operations, elements, components, and/or groups thereof. As used herein, the term "and/or" includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when a feature, such as a layer, region or substrate, is referred to as being "on" another feature or element, it can be directly on the other element or intervening elements may also be present. In contrast, when an element is referred to as being "directly on" another feature or element, there are no intervening elements present. It will also be understood that, when a feature or element is referred to as being "connected" or "coupled" to another feature or element, it can be directly connected to the other element or intervening elements may be present. In contrast, when a feature or element is referred to as being "directly connected" or "directly coupled" to another element, there are no intervening elements present. Although described or shown with respect to one embodiment, the features so described or shown can apply to other embodiments. The term “about” means that the noted number can vary by +/- 20%.

Generally stated, embodiments of the present invention are directed to methods and devices to coat balloons with at least one therapeutic agent using a tube comprising an inner wall providing the at least one therapeutic agent thereon.

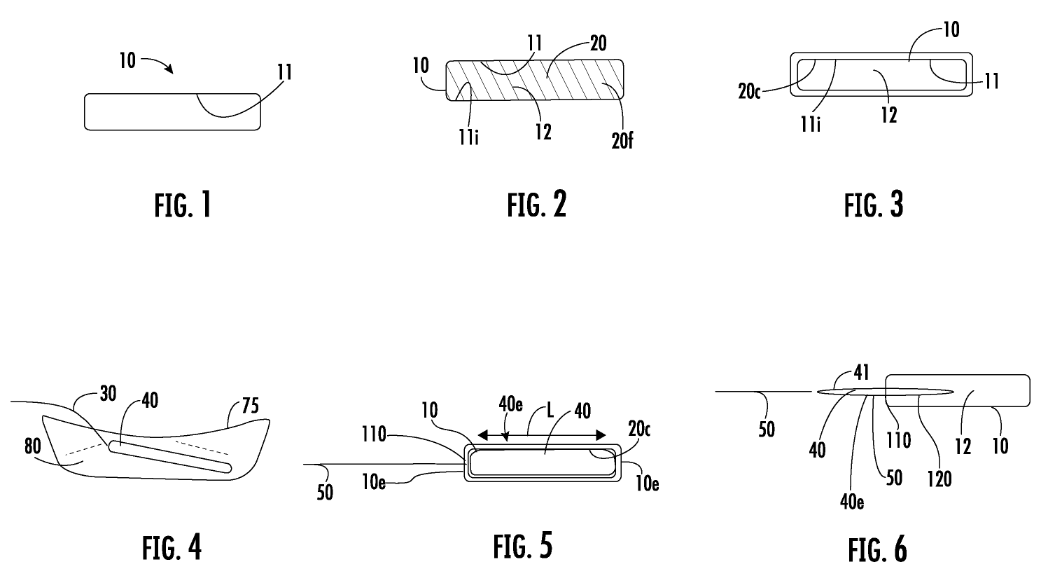

Referring to FIGS. 1-6, an example tube 10 with a wall 11 surrounding a lumen filled with a therapeutic agent 12 (FIG. 2) is shown. The wall 11 of the tube 10 has an inner wall or inner surface 11i with a coating 20c (FIG. 3) comprising at least one therapeutic agent 20. The tube 10 is sized and configured to transfer and/or apply at least some of the coating 20c onto an outer wall 41 of a balloon 40 of a balloon catheter 50 to define a coating 120 comprising the at least one therapeutic agent 20.

The tube 10 can have an inner diameter that can be selected based on the size of the naiive balloon catheter the operator is using. For example, if the operator is using a 6 mm naiive balloon catheter, the inner diameter of the tube can be up to 10% less than 6 mm. In certain embodiments, the tube inner diameter can be in a range of about 5.4 mm - 6.00 mm for a naiive balloon catheter 50 having an expanded nominal outer diameter of about 6 mm. This range is dependent on the elasticity of the tube. A smaller inner diameter of the tube 10 relative to the nominal diameter of the naiive balloon 40 can facilitate complete contact of the outer surface of the naiive balloon with the coating 20c of the tube as the operator deploys the naiive balloon to its nominal size.

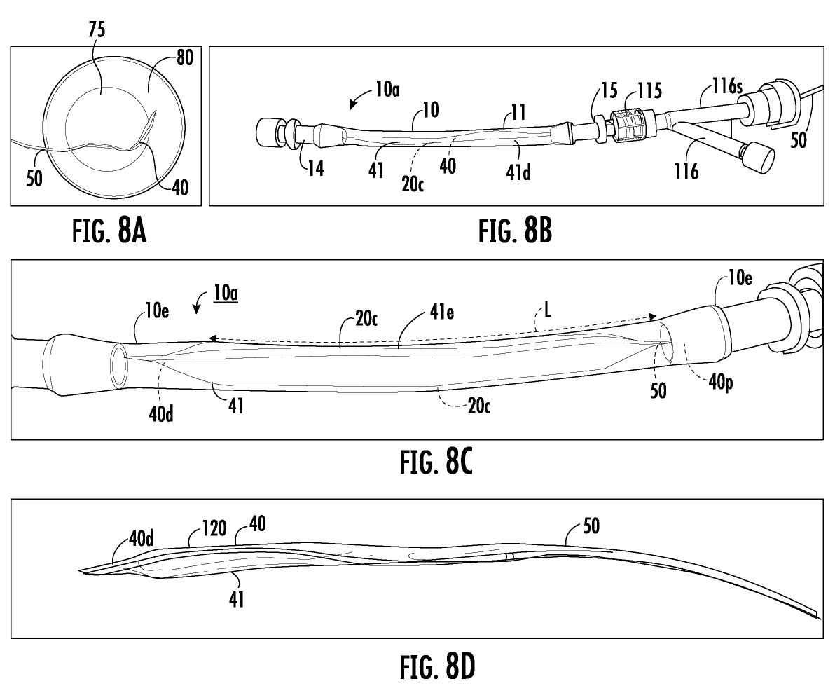

Referring to FIG. 5, the balloon 40 of a medical (balloon) catheter 50 is placed in the lumen 12, in a partially or fully deflated state, 41d, FIG. 8B. The balloon 40 is then expanded to a fully inflated/expanded state 41e (FIGS. 5, 8C) so that the outer wall 41 of the balloon 40 contacts the inner surface 11i of the wall 11 for a defined time period to transfer at least one therapeutic agent 20 from the inner surface 11i of the tube 10 to the outer wall 41 of the balloon 40 to form the coating 120 of the at least one therapeutic agent 20 on the outer wall 41 of the balloon.

The term “transfer” means that the substance is deposited or otherwise applied to the target surface, e.g., the inner surface 11i of the tube 10, then to the outer surface 41 of the balloon 40. The term “deposit”, and derivatives thereof, is used broadly and encompasses any manner of providing the coating on the tube inner surface or the balloon outer surface.

The tube 10 can be elastic. The tube 10 can have a compliant body that mimics a natural (human and/or pig) arterial wall. The balloon 40 can be configured to expand to be larger than an inner diameter of a target artery by about 10-30%. The tube 10 can be sufficiently compliant and/or elastic to accommodate this expansion. The tube 10 can have a greater wall thickness and/or be less elastic than the balloon 40.

The tube 10 can be configured to have sufficient compliancy/flexibility to be able to expand to a diameter at least 10 % greater than a non-expanded state and can comprise any suitable material. The tube 10 can comprise synthetic materials such as silicone, polyurethane, latex, or other polymers and/or copolymers. The tube 10 can comprise biological materials such as decellularized tissue or collagen or electrospun tissue or even plant-based materials. The tube 10 can be 3-D printed or molded or otherwise fabricated. Combinations of the different materials may also be used.

In certain embodiments, the tube 10 can be inelastic, rigid or substantially rigid so as to not expand when the balloon 40 is expanded inside the tube 10.

In certain embodiments, the tube 10 can be formed of silicone such as SYLGARD 184.

The tube 10 can be autoclaved or ethylene oxide (ETO) sterilized for medical grade sterility. The inner surface 11i of the tube 10 can be smooth or have a texture and/or patterned surface to facilitate drug deposition thereon and/or drug transfer therefrom. The inner surface 11i can be free of other chemicals or comprise other layers and/or coatings between the inner surface 11i and the coating 20c.

The coating 120 can extend longitudinally and circumferentially about at least a major portion of a length and circumferentially about the outer wall 41, typically, after removal from the tube 10, the coating 120 extends about an entire circumference of the outer wall 41 and along a length of the expandable part 40e of the balloon 40, based on the contact with the inner wall 11i of the tube 10.

The term “therapeutic agent” refers to any substance used to treat a desired condition and/or subject. The term “therapeutic agent” can be used interchangeably with “drug”. "Treat," "treating" or "treatment of" (and grammatical variations thereof) as used herein refer to any type of treatment that imparts a benefit to a subject and may mean that the severity of the subject’s condition is reduced, at least partially improved or ameliorated and/or that some alleviation, mitigation or decrease in at least one clinical symptom associated with delivery and/or administration of the therapeutic agent is achieved and/or there is a delay in the progression of the symptom. The therapeutic agent can be provided as one drug, multiple drugs, one drug with a drug carrier (not necessarily a drug), or multiple drugs with multiple carriers. A therapeutic agent may be formulated for administration in a pharmaceutical carrier in accordance with known techniques. See, e.g., Remington, The Science and Practice of Pharmacy (9th Ed. 1995), the contents of which are hereby incorporated by reference as if recited in full herein. In the manufacture of a pharmaceutical formulation according to the invention, a therapeutic agent (including a physiologically acceptable salt thereof) is typically admixed with, inter alia, an acceptable carrier. The carrier must, of course, be acceptable in the sense of being compatible with any other ingredients in the formulation and must not be deleterious to the subject. The carrier may be a solid or a liquid, or both, and may be formulated with a therapeutic agent as a unit-dose formulation, for example, a solution (e.g., that may dry to form a coating) and/or powder, which may contain from 0.01 or 0.5% to 95% or 99% by weight of the therapeutic agent. One or more therapeutic agents may be incorporated in the formulations of the invention, which may be prepared by any of the well-known techniques of pharmacy comprising admixing the components, optionally including one or more excipients.

The therapeutic agent can comprise any anti-proliferative drug, anti-inflammatory drug, anti-migratory drug, any excipient, vitamin, protein, cell, or other substance, including combinations thereof. In some embodiments, a therapeutic agent may inhibit and/or block restenosis of an artery. In some embodiments, a therapeutic agent may inhibit and/or block restenosis of a vein. In some embodiments, a therapeutic agent may inhibit and/or block restenosis of an either a stented or non-stented arterio-venous fistula or arterio-venous bypass graft. In some embodiments, a therapeutic agent may inhibit and/or block restenosis of a urethra.

In some embodiments, a therapeutic agent and/or formulation of the invention comprises paclitaxel and/or sirolimus. Embodiments of the invention are particularly suitable for inter-arterial delivery of a therapeutic agent. The therapeutic agent can be configured or selected to target one or more of proliferation, migration and inflammation and the therapeutic agent can be coated, optionally in a therapeutically effective amount, onto the inner surface of the tube 10 and transferred to an outer wall 41 (which can also be interchangeably described as an “outer surface”) of a balloon 40. As used herein, the term "therapeutically effective amount" refers to an amount of a therapeutic agent that elicits a therapeutically useful response in a subject. Those skilled in the art will appreciate that the therapeutic effects need not be complete or curative, as long as some benefit is provided to the subject.

The therapeutic agent can be configured to inhibit and/or block restenosis of an artery. Suitable example therapeutic agents include, but are not limited to, paclitaxel, sirolimus (including taxol or limus derivatives), everolimus, biolimus, zotorolimus, limus drugs, and nab, which is an albumin-bound paclitaxel. However, these are merely examples as there are many other therapeutic agents as will be known to those of skill in the art.

In some embodiments, the at least one therapeutic agent 20 can include certain types of advantageous cells that act as vaccines or other medicaments (for example, antigen presenting cells such as dendritic cells). The dendritic cells may be pulsed with one or more antigens and/or with RNA encoding one or more antigen. Exemplary antigens are tumor-specific or pathogen-specific antigens. Examples of tumor-specific antigens include, but are not limited to, antigens from tumors such as renal cell tumors, melanoma, leukemia, myeloma, breast cancer, prostate cancer, ovarian cancer, lung cancer and bladder cancer. Examples of pathogen-specific antigens include, but are not limited to, antigens specific for HIV or HCV. The therapeutic agent can include other cell-based medicaments, including stem cell medicaments.

In some embodiments, the at least one therapeutic agent 20 can be to treat one or more of erectile dysfunction (internal pudendal arteries), venous stenosis/disease, central airway obstruction, urethral stricture disease, and lower urinary tract symptoms. Studies have been made showing that these conditions can be treated using drug coated balloons.

The at least one therapeutic agent 20 can be provided and applied to the inner surface 11i of the tube 10 in any suitable manner to provide the coating 20c.

The at least one therapeutic agent 20 can be provided to the tube 10 in a liquid solution to provide the coating 20c.

The at least one therapeutic agent 20 can be provided to the tube 10 in a liquid solution by using a dipping method to provide the coating 20c.

The at least one therapeutic agent 20 can be provided to the tube 10 in a liquid solution by using a micro-pipetting method to provide the coating 20c.

The at least one therapeutic agent 20 can be provided as a gas or gas-liquid mixture or liquid (solution) and applied by filling the lumen 12 with the gas or gas-liquid mixture or liquid, or by vapor deposition, spray, micropipette application, a solvent-free electrostatic coating process or other methods/processes to form the coating 20c.

If more than one therapeutic agent 20 is provided, they can be mixed together and applied as a mixture or serially applied, one over another, to the inner wall 11i.

The at least one therapeutic agent 20 can be provided as a liquid solution formed by mixing a liquid formulation, a solid particle formulation, a micro- or nano-particle formulation, or a powder formulation of the at least one therapeutic agent with a liquid diluent or carrier to provide a liquid solution for depositing the therapeutic agent on the inner surface 11i of the wall 11 of the tube 10.

The at least one therapeutic agent 20 can be provided as liquid mixture with a diluent and/or carrier solution to provide a liquid mixture of the at least one therapeutic agent for coating the inner surface 11i of the wall 11 of the tube 10. The diluent or carrier liquid can comprise ethanol, but other liquids may also be used, alone or in combination with ethanol.

The at least one therapeutic agent 20 can be provided in a fluid mixture 20f in an amount sufficient to fill, optionally expand, the tube 10 to form a layer or film on the inner wall 11i of the tube 10. Excess amounts can be removed. This layer or film can be dried to form the coating 20c. This layer or film can remain “wet” to provide the coating 20c.

In some embodiments, the at least one therapeutic agent 20 can be provided in a solution comprising a carrier or diluent.

As shown in FIG. 2, a fluid 20f mixture with the at least one therapeutic agent 20 can be provided to at least partially fill the lumen 12 of the tube 10. In some embodiments, the tube 10 with the fluid mixture 20f can fill an entire volume of the lumen and incubated at room temperature or other temperature for a defined time, such as 12-72 hours, typically at least about 24 hours. Then, as shown in FIG. 3, the fluid mixture 20f can be removed and/or released, leaving a layer of the fluid mixture 20f on the inner surface 11i. If the fluid mixture 20f is provided as a liquid mixture, excess liquid can be drained from the lumen 12.

The inner surface 11i with some of the fluid mixture 20f deposited thereon can be allowed to dry for a defined time, such as 1-48 hours, typically at least 2 hours. The drying can be passive drying by exposing the lumen 12 to ambient temperature. The drying can be active by heating the tube 10 in an oven or using a heat gun. The drying can be active by flowing ambient air and/or heated gas into and/or through the lumen 12.

As discussed above, other methods can be used to deposit the at least one therapeutic agent on the inner surface of the tube 10. The at least one therapeutic agent 20 can be deposited on the inner surface of the tube 10 using one or more of vapor deposition, spraying, micropipette application, a solvent-free electrostatic coating process or other methods/processes to form the coating 20c.

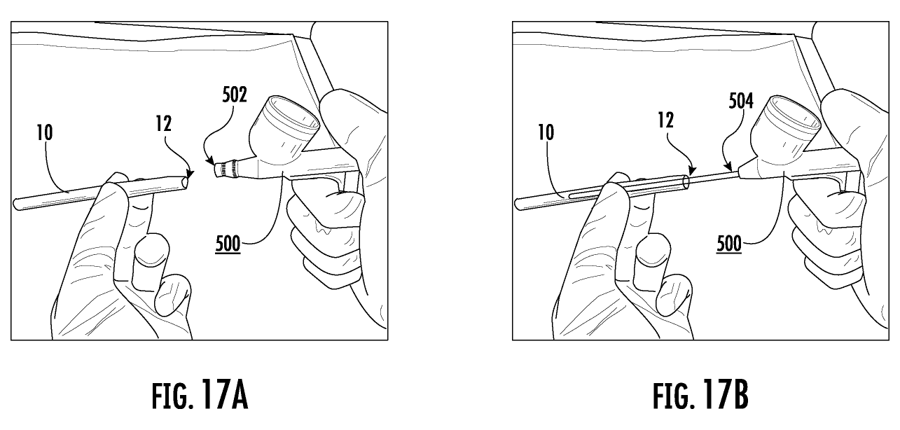

FIG. 17A illustrates a spray coating system 500 with a nozzle 502 aligned with the lumen 12 of the tube 10, and positionable to be adjacent and external and/or internal to the tube 10, can be used to spray at least one therapeutic agent into the tube 10 and coat the inner surface 11i. The spray coating system 500 can be set to operate at any suitable pressure to provide a suitable spray pattern. The nozzle 502 can remain external to the tube 10 during the spray coating. The nozzle 502 can be inserted into one end or into each end of the tube, serially, to deposit the coating. To achieve coating, in some embodiments, the spray coating can be attached to an air compressor, pressure regulator, and a multi-purpose high performance airbrush. The spray coating system 500 can comprises a fluid source container. In some embodiments, about a one third ounce fluid cup and 0.2 mm tip can be used. The pressure regulator can be set to a desired pressure, such as about 50 psi. However, other pressures may be used. The spray tip of the nozzle 502 can be spaced about 1-2 inches from the entrance of the tube 10 and the therapeutic agent can be sprayed for about 10 -20 seconds, equivalent to about 4-5 ml of therapeutic agent. The process can be repeated for the other end of the tube 10 and the tube with the applied coating can be allowed to dry for a suitable time, such as 8-24 hours, or overnight.

As shown in FIG. 17B, instead of a nozzle, a spray bar 504 (which can also be interchangeably referred to as a “spray tube”) can be coupled to the spray coating system 500 and be sized and configured to enter the tube 10 and spray the mixture with at last one therapeutic agent onto the inner surface 11i of the tube 10. The spray tube 504 can be straight as shown in FIG. 17B or can be shaped such as curvilinear. The spray bar 504 can be slidably extended as the spray bar sprays and can be rotated in the tube 10 to apply the coating, as desired. The spray bar 504 can have a lumen enclosed by a closed wall that directs the spray out the end or may have one or more outwardly facing apertures in the wall, such as a plurality of axially spaced apart and/or circumferentially spaced apart apertures. The spray bar 504 can be operated manually or using an automated system with an electro-mechanical handling system such as a robotic interface.

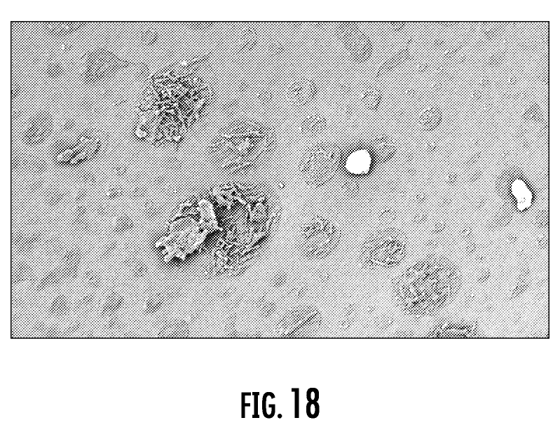

FIG. 18 is a photograph showing 12 mg/ml of dissolved solid paclitaxel spray coated onto the inner surface of the tube 10 using the system shown in FIG. 17A.

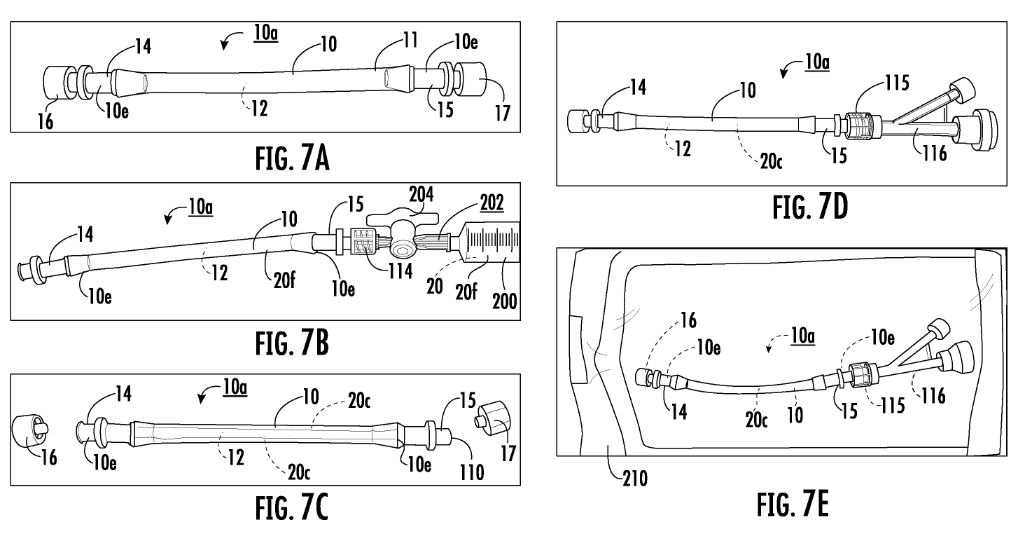

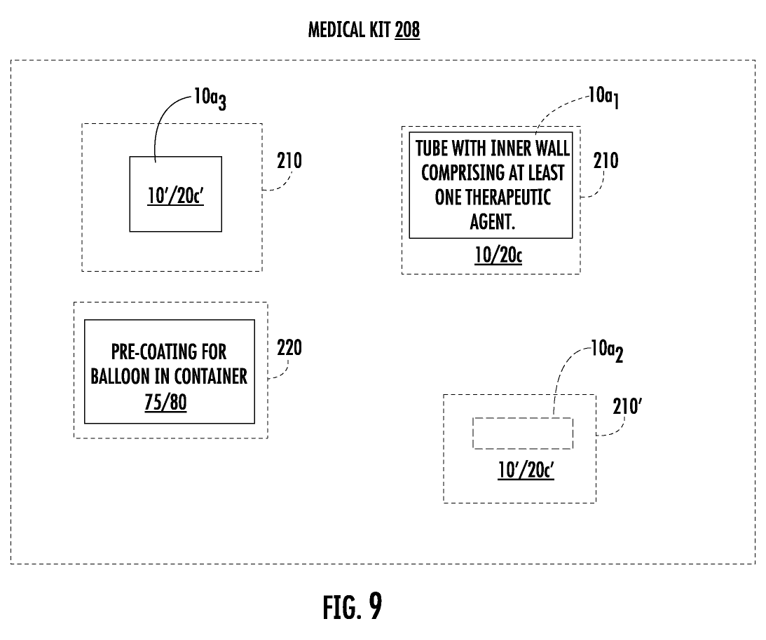

The tube 10 with the coating 20c can be packaged and sterilized to medical sterility standards for medical procedures, before, during or after placement in packaging 210 (FIGS. 7E, 9), via ETO or other sterilization methods.

As discussed above with respect to FIG. 5, the balloon catheter 50 with a balloon 40 can be placed in the lumen 12, the balloon 40 expanded to have the outer wall 41 at an expanded state 41d to press outward against the inner surface 11i of the tube 10 for a defined time that is typically in a range of 1-10 minutes, typically in a range of 1-5 minutes, such as about 1 minute, about 2 minutes, about 3 minutes, about 4 minutes or about 5 minutes or any increment therebetween. During the drug transfer, the outer wall 41 of the balloon 40 can be (over) expanded up to about 30% of a diameter of the tube 10 to maximize surface contact between the outer surface 41 of the uncoated balloon 40 to the inner surface 11i of the (drug-lined elastic) tube 10, to aid in drug transfer. The coating 120 is thus applied to the surface of the outer wall 41.

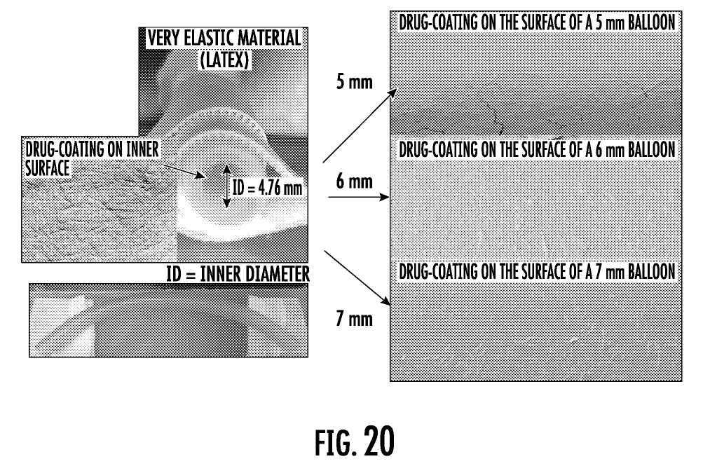

In some embodiments, a single drug-lined elastic tube 10 can be configured to be able to coat a plurality of different size (expanded OD and/or length) balloons 40, such as 5 mm, 6 mm and 7 mm (expanded OD size) balloons 40 of a respective balloon catheter 50. This is due to the selected elasticity of the drug-lined tube 10. A sufficiently elastic tube 10 can therefore be selected to provide a drug-lined elastic tube 10 that can coat and accommodate different size balloons 40 of the balloon catheters 50. The ability to use a single size (ID) tube 10 to coat different size balloons 40, both in length and diameter, can provide an economical benefit for purchasing and storage purposes. Health care providers do not need to purchase a plethora of sizes to be able to have the one needed for a particular use. FIG. 20 shows an example single-size drug-lined tube 10 of latex that can coat outer surfaces of three different OD size balloons 40 (SEM images on the right show the coatings on the outer surface).

As shown in FIG. 5, the tube 10 can be elongate with opposing end portions 10e, at least one of which provides a port 110 to slidably receive/enter the balloon 40 of the balloon catheter 50. The port 110 can be provided with a fitting.

As shown in FIG. 6, the balloon 40 with the coating 120 on the outer wall 41 can be deflated and removed from the tube 10 via the port 110. The balloon 40 with the coating 120 on the surface of the outer wall 41 can now be used for a medical procedure to deliver the at least one therapeutic agent 20 provided by the coating 120 to target tissue. No drying or time delay is required prior to insertion into a patient for delivering the at least one therapeutic agent 20 provided by the coating 120.

As shown in FIGS. 4 and 8A, the balloon 40 can be pre-coated with a liquid 80 such as an excipient, optionally a contrast agent placed in a container 75. The pre-coating can be carried out at the clinical site of the medical procedure or the balloon 40 can be pre-coated at an OEM site or other preparation site and provided in a sterile package for use onsite.

The catheter 50 with the balloon 40 can be an off the shelf “uncoated” (meaning not coated with a therapeutic drug) angioplasty catheter balloon. However, the methods and systems can be used for other balloon-based medical therapy devices.

In some embodiments, excess liquid from a liquid solution providing the at least one therapeutic agent 20 can be removed from the lumen 12 of the tube 10, leaving the coating 20c on the inner surface 11i of the wall 11 of the tube 10 with the remainder of the lumen 12 free of the at least one therapeutic agent 20 as shown in FIG. 2.

The amount of therapeutic agent 20 deposited onto the inner surface 11i can be in any suitable amount to be able to subsequently transfer a therapeutic dose to the outer surface 41 of the balloon 40 at least once for a respective medical procedure. In some embodiments, the amount on the outer surface of the balloon 40 transferred by the tube 10 can be in a therapeutically effective amount which may be in a range of 0.01 μg/mm² to 10 μg/mm² or greater.

The amount of the therapeutic agent 120 on the outer wall 41 of the balloon 40, provided by the coating 20c of the tube 10, can be in any suitable amount to be able to deliver a therapeutic dose to target tissue during a medical procedure with the balloon 40 expanded so that the outer wall 41 presses outward against and contacts the target tissue for a suitable time, typically in a range of 1-10 minutes, such as about 2 minutes, to deliver a therapeutic dose of the at least one therapeutic agent 20 to target tissue. In some embodiments, measured 1 hour post-delivery by an ex vivo model or animal study, the delivered amount can be in a range of 0.01 ng/mg to 100 ng/mg or greater.

The method of drug delivery is critical, as very slow or very fast delivery may result in subtherapeutic levels. Thus, the therapeutic agent 120 can be released from the balloon 40 to local tissue over a relatively short inflation time period, e.g., 1-5 minutes, with a balloon-to-artery ratio of about 1.1 to 1.3 in a reliably repeatable manner based on the coating 20c from the tube 10.

For arterial paclitaxel, the therapeutic dose range provided to the luminal artery wall by the coating 120 on the balloon 40 can be in a range of 0.01 to 100 ng/mg (paclitaxel amount/weight of arterial tissue)²⁵. See, Axel DI, Kunert W, Goggelmann C, Oberhoff M, Herdeg C, Kuttner A, Wild DH, Brehm BR, Riessen R, Koveker G, et al. Paclitaxel inhibits arterial smooth muscle cell proliferation and migration in vitro and in vivo using local drug delivery. Circulation. 1997;96:636-645; the contents of which are hereby incorporated by reference as if recited in full herein.

Referring now to FIGS. 7A-7E, an example tube assembly 10a sized and configured for coating a balloon 40 of a balloon catheter 50 according to embodiments of the present invention is shown. The tube assembly 10a is elongate and has opposing ends 10e, each with small bore connectors, such as luer fittings 14, 15, respectively, and one or both of these luer fittings 14, 15 can be valved luer fittings 14, 15. Releasable end caps 16, 17 can attach to respective fittings 14, 15 be used to sealably close the lumen 12.

In some embodiments, which may be clinically desired, is that the tube assembly 10a can be configured as a closed system. This way when the physician/clinician uses this tube assembly product and coats the balloon, it can ensure that any remaining drug is either coated on the balloon or remains in the closed system, minimizing drug contamination in the medical area such as the surgical room.

FIG. 7B shows the end cap 17 replaced with a connector 114. The tube assembly 10a is coupled to a syringe 200 via the connector 114 coupled to fitting 15. The syringe 200 comprises a fluid mixture 20f comprising the at least one therapeutic agent 20. The syringe 200 can force the fluid mixture 20f into the lumen 12 to fill the lumen 12 of the tube 10 according to embodiments of the present invention. As shown, the syringe 200 can be coupled to a small length of flexible conduit 202 positioned between the threaded connector 114 and the tip of the syringe 200. An external valve 204 can be used to control flow of the fluid mixture 20f. The end cap 16 can remain in position to fill the lumen 12 or it can be removed and the fluid mixture 20f delivered through the lumen 12 in sufficient volume to fill the lumen 12 to circumferentially and longitudinally “L” along at least a major length of the tube 10, typically an entire length to coat the entire interior wall 11i.

FIG. 7C shows the tube assembly 10a, post-fill, with the syringe 200 and connector 114 removed and the end caps 16, 17 also removed allowing the fluid mixture 20f on the inner wall 11i of the tube 10 to dry to form the coating 20c according to embodiments of the present invention.

FIG. 7D shows the tube assembly 10a with the coating 20c and a connector 115 having a Y segment 116 attached to the tube assembly 10a in place of end cap 17. FIG. 7E shows the tube assembly 10a with the coating 20c and the connector 115 on one end portion 10e of the tube 10 and the end cap 16 on the other end portion 10e of the tube, all assembled together and placed in packaging 210. The packaging 210 can be a sterilization pouch. The assembled components can be sterilized in the packaging 210 or before being placed in the packaging, typically using ETO as will be understood by those of skill in the art.

FIGS. 8A-8D show a sequence of actions that may be used for coating the balloon 40 (angioplasty) according to embodiments of the present invention. FIG. 8A shows the (uncoated) balloon 40 (angioplasty) positioned into a container 75 comprising a liquid 80 which can comprise an excipient such as contrast agent. This “pre-coats” the outer wall 41 of the balloon 40 to facilitate drug transfer from the coating 20c of the inner wall 11i of the tube 10. In some embodiments, no drying time is required before inserting the “pre-coated” balloon 40 into the tube 10. However, it is contemplated that a short passive (air dry) or active heat assisted drying time, such as 1-5 minutes, may be used.

In certain embodiments, a physician may directly go from using a balloon to open a blocked artery to then coating that balloon with the at least one therapeutic agent provided by the tube 10 and bypassing the precoating of the balloon in the contrast (FIG. 8A). This is because when the balloon has been used in the patient, this can essentially pre-coat the balloon with blood and other ‘proteins’ from the wall of the patient.

FIG. 8B shows the balloon 40 of the balloon catheter 50 being placed within the lumen 12 of the tube 10 in a fully or partially deflated state 41d. A distal end 40d of the balloon 40 resides adjacent one end portion 10e of the tube 10 and a proximal end portion 40p resides adjacent the other end portion 10e of the tube. The placement can be carried out using a connector, optionally one leg of the Y connector segment 116, typically the leg segment 116s that is in-line with the lumen 12. However, it is noted that the Y-connector configuration is not required and other connector/fitting configurations may be used and a respective connector/fitting can be valved. FIG. 8C shows the balloon 40 is then expanded to an expanded state 41e within the tube 10. FIG. 8D shows the resulting balloon 40 with the coating 120 on the outer wall 41, deflated and removed from the tube 10 (and connector 115) and ready to be positioned and deployed within an artery.

The therapeutic agent 120 can be transferred from the balloon outer wall surface 41 to a targeted arterial tissue at a therapeutic level. Once the balloon catheter is positioned in the artery at a first target site and the at least one therapeutic agent from the coating 120 transferred thereto, the balloon catheter 50 can be removed from the patient/subject, then inserted into another tube assembly 10a for reapplying the coating 120 and the balloon catheter 50 can be redeployed into the patient to treat a different target site thereby reusing the balloon 40 multiple times during a single medical procedure. Thus, at least first and second tube assemblies 10a can be serially used for a single balloon catheter 50 for any respective single medical procedure. The first tube assembly 10a can comprise the same coating 20c with the same at least one therapeutic agent in the same concentration or a different at least one therapeutic agent in the same or different concentration.

In certain embodiments, the clinician can use a new balloon catheter 50 for each reinsertion or for some of the reinsertions with the same tube assembly 10a or a new tube assembly 10a for providing a desired therapeutic agent from a coating 120.

DCBs have shown to be effective in various arterial disease including peripheral arterial disease¹⁰, dialysis access¹¹, coronary artery disease¹² and in-stent restenosis¹³. There has been a wide variety of DCBs manufactured with various drug concentration, pharmacokinetic profile and various drug coating types (drug-excipient combination)¹⁴. However, to date, all DCBs share one common attribute; uncoated balloon angioplasty balloons are coated and packaged at the manufacturing site and shipped to the operating room. And once the DCB is used, the balloon is trashed.

The early DCB was configured to treat peripheral arterial disease due to suboptimal performance of uncoated balloons and stents to maintain patency following intervention¹⁵. The first two FDA approved DCB, Lutonix DCB and IN.PACT, were able to show clinically efficacy in the treatment of femoropopliteal artery disease. The Lutonix DCB utilizes low dose paclitaxel (2 µg/mm²) on a platform of polysorbate and sorbitol¹⁶. The IN.PACT Admiral utilizes high dose paclitaxel (3.5 µg/mm²) coated with urea as its carrier¹⁷. The pivotal LEVANT 2 trial facilitated the FDA approval for Lutonix¹⁸. Using a randomized trial of 476 patients with symptomatic femoropopliteal artery disease, patients treated with the Lutonix DCB (versus standard PTA), demonstrated significantly better outcomes in primary patency at 12 months (65.2% vs. 52.6%, P=0.02), driven by a reduction in binary restenosis. Similarly, the IN.PACT SFA multicenter, randomized controlled trial of 331 patients showed significantly higher rates of freedom from restenosis in patients treated with the IN.PACT DCB as compared to standard PTA (82.2% vs. 52.4%, P<0.001)¹⁹. Additionally, clinically driven TLR was significantly lower with DCB compared to standard PTA (2.4% vs. 20.6%, P<0.001).

In addition to Lutonix and IN.PACT DCB, two additional DCBs have also received FDA approval, including Stellarex DCB (Phillips, Colorado Springs, CO) in July 2017 and Ranger DCB (Boston Scientific Marlborough, MA). The Stellarex DCB utilizes low dose paclitaxel (2 µg/mm²) coated with polyethylene glycol as the carrier through the proprietary technology, EnduraCoat^{TM} . The Ranger DCB with a paclitaxel dose of 2 µg/mm² on the market that uses a citrate ester (acetyl-tributyl citrate) as the excipient coated with proprietary technology TransPax^{TM}. More recently, The SurVeil^{TM} DCB (Surmodics Inc.) was FDA approved in June 2023. The SurVeil DCB is coated with a paclitaxel dose of 2 µg/mm².

In addition to the treatment of PAD, DCBs have also been utilized in the treatment of arteriovenous fistula (AVF) stenosis. Hemodialysis access dysfunction among end-stage renal disease patients costs approximately $3 billion annually within the US Medicare population alone. The most common cause of access dysfunction is venous stenosis caused by neointimal hyperplasia, for which percutaneous transluminal angioplasty is the first-line treatment. The IN.PACT AV investigational device exception (IDE) 6-month study was published in 2020²⁰ with a 12-month outcomes study published in 2022²¹. Using 330 participants across three countries – the U.S., Japan, and New Zealand – treatments were randomly assigned into the either PTA group (n = 160) or the DCB group (n = 170). Results demonstrated that at 6 and 12 months, the IN.PACT DCBs were superior to PTA (6-months: 82.2% DCB vs 59.5% PTA, p < 0.001; 12-months: 63.8% DCB vs. 43.6% PTA, p < 0.001). The Lutonix AV IDE 180-day study for the Lutonix DCB was published in 2018²² with a 2-year outcomes study published in 2020²³. Of the 285 subjects in the study, 141 were randomly assigned to the DCB group and 144 to the PTA group. At months, no significant difference was found between the DCB and PTA groups (71% DCB vs. 63% PTA, p = 0.06). However, at 9- and 12-months, the DCB showed superiority over the PTA (9-months: 58% DCB vs 46% PTA, p = 0.02; 12-months: 44% DCB vs. 36% PTA, = 0.04).

The biggest hurdle with the wide usage of DCB is the cost. With the expiration of Medicare’s transitional pass-through payment in 2018, providers now receive no additional reimbursement for using a DCB⁹.

Additionally, for periphery and dialysis treatment, disease can be widespread requiring multiple DCBs, which further exaggerates the cost consideration component. To this end, some DCB manufacturing companies have developed very long (150 and 300 cm length) DCBs to reduce the need for multiple DCBs, however this has its own complications. These longer DCBs are deployed in tapered vessels and thus the ideal balloon-to-artery ratio cannot typically be attained, which limits the efficacy of these DCBs.

In recent years, other devices have been introduced to the market to provide an alternative to DCBs, mainly liquid delivery devices²⁴. These devices are able to deliver therapy in liquid form and can treat multiple sites within the vasculature. However, these devices are also expensive ($1,000 to $2,000), require training and also added supporting devices. In addition, the treatment time of these devices are longer than a DCB given that the delivery catheter needs to be preloaded with the liquid therapy.

Embodiments of the present invention can provide many benefits. First, the coating method can be used with the balloon angioplasty that the operator is already using during the procedure, saving cost. Second, the operator can maintain all their preferred supporting endovascular wires and catheters. Third, the operator can be more strategic and re-coat the angioplasty balloon 40 to treat multiple treatment sites during a single medical procedure. Currently, the operator maybe limited to only one lesion to treat (due to cost consideration) or to use a very long DCB, in hope of covering most of the target area with one balloon. Fourth, there is little to no training required. The approach to coat the balloon 40 is the same approach the operator deploys a balloon 40 in the artery. Therefore, no special training is required.

The tube 10 can be elastic tubing to provide the coating apparatus at the surgical site. An elastic tubing, with diameter compliance similar to an artery is useful or multiple reasons. First, the elasticity avoids damage to the (uncoated) balloon 40 during deployment within the tube 10. A rigid or semi-rigid tube may damage the balloon 40 as it is inflated within the tube 10. Second, to aid in drug transfer, the elasticity can be selected to provide sufficient surface interaction of the drug-lined tube 10 with the surface of the outer wall surface 41 of the balloon 40, at a fully or even over expanded inflation state, such as at a maximum inflation state.

However, in certain embodiments, the tube 10 is not required to be elastic and may be inelastic or rigid as discussed above.

The tube 10 can be visually transmissive, such as formed of a clear material which allows the operator to be able to visually view the balloon 40 through the tube 10 as the balloon 40 is being coated within the tube 10.

The balloon 40 in the tube 10 can be expanded and deployed typically in a range of about 30 seconds to about 10 minutes, such as about 30 seconds, about 45 seconds, about 1 minute, about 2 minutes, about 3 minutes, about 4 minutes, about 5 minutes, about 6 minutes, about 7 minutes, about 8 minutes, about 9 minutes or about 10 minutes, optionally with a balloon 40 to tube 10 ratio of 1.1 to 1.3:1.0.

The described approach also has other potential benefits. The drug-lined (elastic) tube 10 can be configured to coat any single or multiple drug combination with and without excipients on the inner surface 11i.

It is contemplated that concentration of the at least one therapeutic agent (drug) can be modified to increase or decrease the amount of drug loaded onto the (uncoated) balloon 40 (angioplasty) by the tube 10 for different diseases, target sites and/or patients.

FIG. 9 illustrates an example medical kit 208 comprising at least one tube 10 with the coating 20c, which may be placed in a package 210. The kit 208 typically does not include a balloon catheter 50 with a balloon 40, which may be provided separately. The kit 208 may also include a pre-coating substance 80 optionally with a container 75 for applying the pre-coating to the outer surface of the balloon 40. Optionally, the kit 208 may comprise at least one additional tube 10’ with a respective coating 20c’ in a package 210’. The at least one additional tube 10’ can have the same or different coating 20c’ as the coating 20c of the first tube 10 with its coating 20c. If the same coating, it may be in a different dosage amount thereby allowing an operator to select which to use at the clinical site for a particular patient and/or target site. As shown, the kit 208 may also include another additional tube (a third tube) 10’ with coating 20c so that the kit 208 comprises three tube assemblies 10a₁, 10a₂, 10a₃ with respective coatings 20c for use to serially coat a single balloon catheter 50 during a single medical procedure of a respective patient.

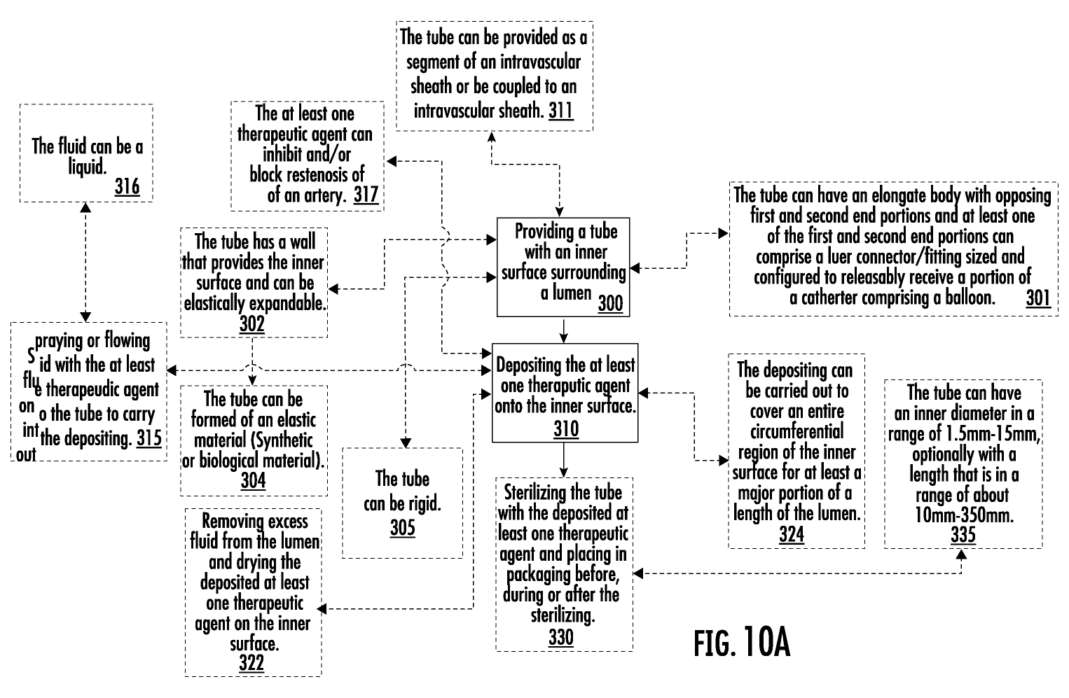

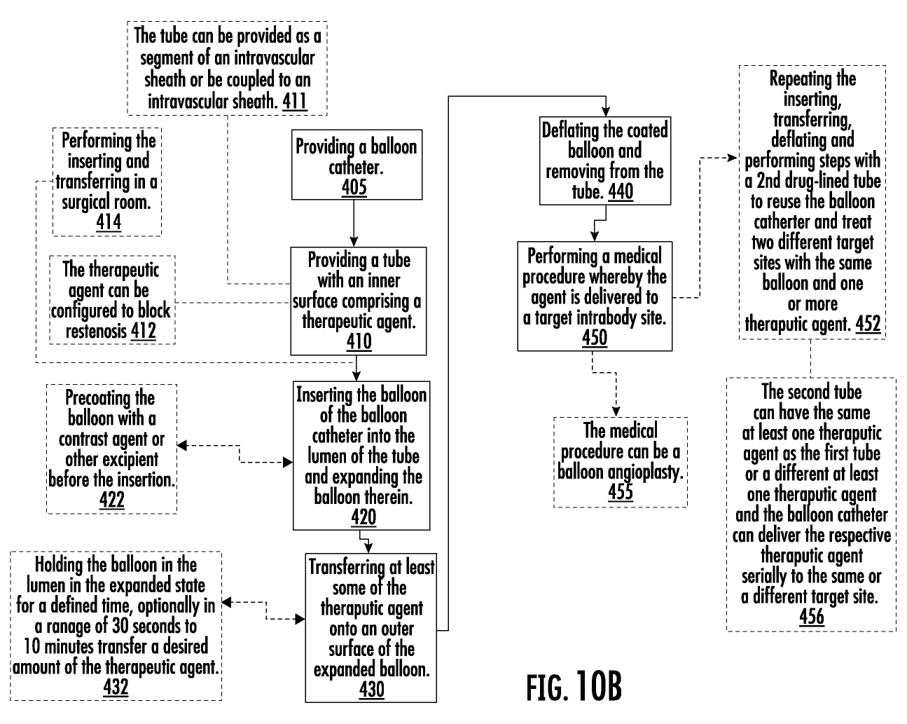

Turning now to FIG. 10A, example actions are shown that can be used to produce a tube with a coating. A tube with an inner surface surrounding a lumen is provided (block 300). At least one therapeutic agent is coated (deposited) onto the inner surface of the tube (block 320).

The tube with the deposited at least one therapeutic agent can be sterilized and placed in packaging before, during or after the sterilizing (block 330).

The tube can have an inner diameter in a range of about 1.5 mm to about 15 mm, optionally with a length that is in a range of about10 mm to about 350 mm (block 335).

The tube can have a wall that provides the inner surface and that is (elastically) expandable (block 302).

The tube can be formed of an elastic material (block 304). The elastic material can comprise synthetic and/or biological material.

The tube can be rigid (block 305) whereby the tube does not expand under normal pressures when contacted by the balloon of the balloon catheter, when expanded.

The coating can be carried out by spraying fluid with the at least one therapeutic agent into the tube or by flowing fluid with the at least one therapeutic agent into the tube (block 315). Other coating methods can be used including vapor deposition and micro pipetting, for example.

Excess fluid, if any, can be removed from the lumen and the coating (deposited at least one therapeutic agent) on the inner surface can be dried (block 322).

The tube can have an elongate body with opposing first and second end portions and at least one of the first and second end portions can comprise a valved luer fitting/connector sized and be configured to releasably receive a portion of a catheter comprising a balloon (block 301).

The tube can be provided as a segment of an intravascular (introducer) sheath or be coupled to an intravascular (introducer) sheath (block 311).

The fluid can be a liquid (block 312).

The therapeutic agent can inhibit, block or prevent restenosis of an artery or a stented artery (block 317).

The fluid can comprise any suitable drug as the at least one therapeutic agent. The liquid can comprise, for example, any anti-proliferative drug, anti-inflammatory drug, anti-migratory drug, any excipient, vitamin, protein, cells, or other substance at least one therapeutic agent that blocks or at least inhibits restenosis of an artery, optionally at least one of paclitaxel or sirolimus or other limus or paclitaxel derivatives or equivalents.

The coating can cover an entire circumferential region of the inner surface for at least a major portion of a length of the lumen (block 324).

Turning now to FIG. 10B, example actions for providing a drug coated balloon for a medical procedure are shown. A balloon catheter is provided (block 405). A tube with an inner surface comprising a therapeutic agent is provided (block 410). The balloon of the balloon catheter is inserted into the lumen of the tube and the balloon expanded therein (block 420). At least some of the therapeutic agent is transferred onto an outer surface of the expanded balloon (block 430). The coated balloon is deflated and removed from the tube (block 440). A medical procedure is performed whereby the therapeutic agent is delivered to a target intrabody site from the coated balloon (block 450). The medical procedure can be a balloon angioplasty procedure (block 455).

It is noted that where a physician has already used a balloon of their choice from a plethora of options they have in the surgical suite to open a clogged artery, advantageously, that physician can then use that same balloon and insert it into the tube 10 to coat it with the drug/therapeutic agent. Then, the physician can insert the coated balloon back into the same location and repeat the procedure, but in this case the balloon will deliver the drug. Importantly, embodiments of the present invention allow the physicians to use whatever balloon they are most comfortable with. With various tortuosity, lengths and other design factors of balloons, physicians become very particular with what balloons they use when dealing with clogged arteries. Embodiments of the present invention allow a physician to coat a balloon of their choice can be important to clinical implementation.

In certain embodiments, to facilitate maximum and/or substantially uniform coverage of the balloon, the balloon of the balloon catheter can be at least partially deflated after a first inflation for coating the balloon, then rotated a partial turn inside the tube and reinflating to contact the inner surface of the tube again, before removing from the tube. After rotating the partial turn, the balloon can be expanded against the inner surface of the tube again whereby at least some of the at least one therapeutic agent can be transferred. This action can position different portions of the outer surface of the balloon at different portions of the inner surface of the tube so that if a segment of the inner surface of the tube may be devoid of the coating or have a reduced amount, then this action can ensure therapeutic agent is transferred onto substantially an entire circumference at a maximum outer diameter, typically over at least a major portion of a length dimension of the balloon.

The tube can be provided as a segment of an intravascular (introducer) sheath or be coupled to an intravascular (introducer) sheath (block 411).

The coated balloon can be delivered over a guide wire through a vascular sheath to the target site in the patient as is well known to those of skill in the art.

The inserting and transferring can be performed in a surgical room (block 414).

The inserting, transferring and deflating steps can be repeated using another tube with an inner surface comprising the same or a different therapeutic agent and/or the same or different concentration thereof, to reuse the balloon catheter and treat the same target site twice or treat two or more different target sites with the same balloon and the at least one therapeutic agent(s) (block 452).

The second tube can have the same at least one therapeutic agent as the first tube or a different at least one therapeutic agent and the balloon catheter can deliver the respective therapeutic agent serially to the same or a different target site (block 456).

The therapeutic agent can comprise a drug that blocks or inhibits restenosis of an artery or stented artery or vein or stented vein or urethra (block 412).

The balloon can be precoated with a contrast agent or other excipient before the insertion (block 422).

The balloon can be held in the lumen in the expanded state for a defined time, optionally in a range of about 30 seconds to about 10 minutes to transfer a desired amount of the therapeutic agent (block 432).

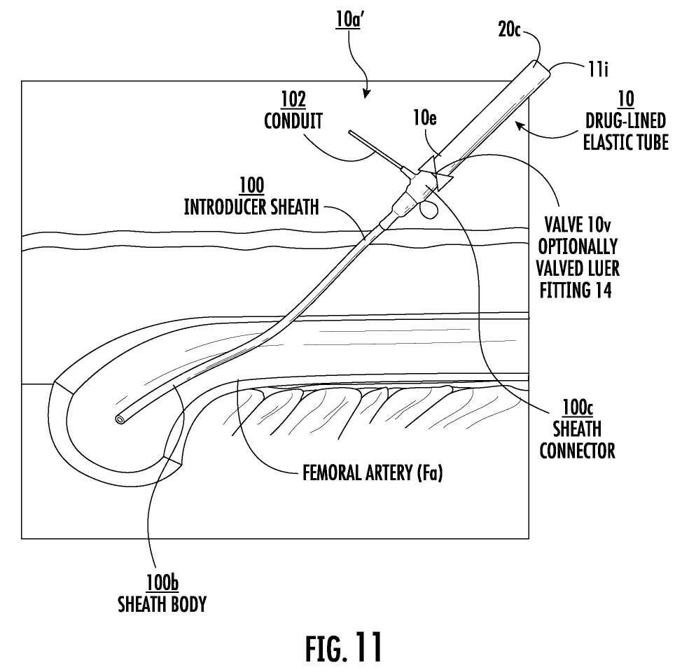

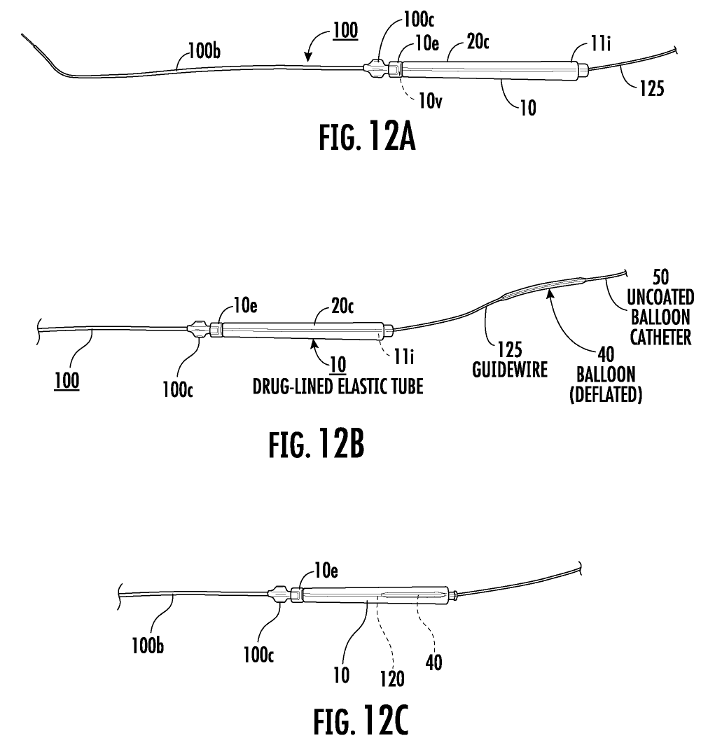

Turning now to FIGS. 11-13, additional embodiments of a drug coated tube are shown.

FIG. 11 is a schematic illustration of a tube assembly 10a’ comprising an intravascular introducer sheath assembly 100 coupled to a tube 10 with an inner surface 11i comprising a coating 20c of at least one therapeutic agent and being sized and configured to coat a balloon 40 of a balloon catheter in situ with the at least one therapeutic agent according to embodiments of the present invention. In this embodiment, the tube 10 can be external to a patient during the coating process which can be performed in situ during a medical procedure with the tube 10 coupled to the intravascular sheath assembly 100 while the sheath body 100b of the intravascular sheath assembly 100 is in vivo inside the patient, optionally at least partially in a femoral artery Fa.

The distal end 10e of the tube 10 can be coupled directly or indirectly to a connector 100c of the intravascular introducer sheath assembly 100. The intravascular introducer sheath assembly 100 can comprise a conduit 102 that branches outward from the sheath body 100b adjacent the connector 100c. The tube 10 can comprise a valve 10v configured to impede blood flow in a proximal direction, into the tube 10, prior to and/or during transfer of the coating 20c to the balloon 40 of the balloon catheter 50.

FIG. 12A is a side view of an example intravascular introducer sheath assembly 100 coupled to the tube 10 with the tube 10 having the inner surface 11i with the coating 20c comprising the at least one therapeutic agent and configured to coat the balloon 40 of the balloon catheter 50. In FIG. 12A, a guide wire 125 extends through the tube 10 and through the intravascular sheath body 100b prior to the balloon being slidably positioned in the tube according to embodiments of the present invention.

FIG. 12B is a side view of the example intravascular introducer sheath coupled to a tube with the tube shown in FIG. 12A, showing the balloon of a balloon catheter proximal to the tube 10 prior to coating and with a guide wire 125 extending through the tube 10 and through the intravascular sheath body 100b prior to the balloon being slidably positioned in the tube according to embodiments of the present invention.

FIG. 12C is a side view of the example intravascular introducer sheath coupled to a tube shown in FIG. 12B, but with the balloon 40 positioned in the tube 10 to apply a coating from the coating 20c on the inner surface, upon at least partial inflation of the balloon 40 in the tube 10. The balloon 40 can be held in the tube 10 for a defined time to carry out the coating to apply the coating 120 to the outer wall 41 of the balloon 40. The defined time can be in a range of 10 seconds to 10 minutes. Then, after the defined time, the balloon 40 with the applied coating 120 can be transitioned back to a deflated state and the balloon 40 and the balloon catheter 50 can be being slidably advanced through the intravascular sheath body 100b to target anatomy whereby the balloon with the applied coating 120 can be inflated to deliver a therapy by transferring a therapeutically effective amount of the applied coating according to embodiments of the present invention.

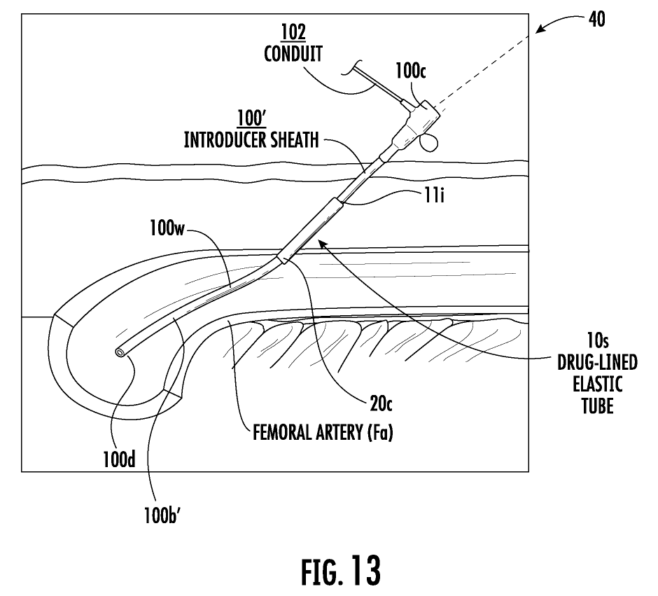

FIG. 13 is a schematic illustration of an intravascular introducer sheath assembly 100’ that has an integrated tube segment 10s configured with an inner surface 11i comprising a coating 20c to coat a balloon 40 of a balloon catheter 40 with at least one therapeutic agent according to embodiments of the present invention. In this embodiment, the tube segment 10s can be internal to a patient during the coating process which can be performed in situ during a medical procedure. The tube segment 10s of the sheath body 100b’ of the intravascular sheath assembly 100 can be in vivo inside the patient, optionally at least partially in a femoral artery Fa, during the coating process to apply a coating 120 on the outer wall 41 of the balloon from the coating 20c with the at least one therapeutic agent. The balloon 40 can be at least partially inflated during the coating for a defined time such as 10 seconds to 10 minutes, while the sheath body 100b of the intravascular sheath assembly 100 is in vivo inside the patient, optionally at least partially in a femoral artery.

The tube segment 10s can be placed inside the sheath body 100b or form a segment of the outer wall 100w of the sheath body 100b. The tube segment 10s can be of the same material as the sheath body 100b’. The tube segment 10s can have increased elasticity with respect to adjacent portions of the sheath body 100b. The tube segment 10s can be positioned closer to the proximal connector 100c than a distal end 100d of the sheath body 100b’.

Embodiments of the invention will be described further below by way of the following non-limiting Examples.

### EXAMPLES

### Methods

### Silicone Elastic Tubing

Silicone elastomer tubes were generated from Sylgard® 184 (Dow Corning, Midland, MI, USA) having an inner diameter of 6.00 mm and total length of 8 cm using 316L stainless steel rods. Briefly, the two-part Sylgard solution was mixed at a ratio of 1:10 and poured over the rotating stainless steel rods. After heating, the tubing was removed and ready for drug seeding.

### Coating of Silicone Elastic Tubing

To coat the inner surface of the elastic tubing, three different solutions were tested. These included: (1) a liquid paclitaxel (Paclitaxel Injection USP, 6 mg/mL, Actavis Pharma, Parsippany, NJ, USA); (2) solid paclitaxel (P-9600 Paclitaxel, LC Labs, Woburn, MA) dissolved in 100% ethanol at a concentration of 6 mg/mL and 12 mg/mL; and (3) solid sirolimus (R-5000 Rapamycin, LC Labs, Woburn, MA) dissolved in 100% ethanol at a concentration of 6 mg/mL and 12 mg/mL. Following luer fitting attachments, the elastic tubing was then filled with the drug solution for 24 hours. After this incubation period, the liquid drug was drained and the elastic tubing air dried. Following the drying of the silicone tubing, luer fittings were attached and packaged for ethylene oxide sterilization (FIGS. 7A-7E).

### Coating of Uncoated Balloon Angioplasty

An uncoated balloon angioplasty was first coated with contrast agent by placing the balloon in a container of contrast agent. The balloon was then positioned into the drug-lined silicone tubing and deployed for 2 minutes at a balloon to silicone tube ratio of 1.1:1.0.

### Ex Vivo Bioreactor System

Porcine carotid arteries from large pigs (250–350 lbs.) were harvested from a local abattoir. The arteries were transferred in sterile PBS with 1% antibiotic-antimitotic (Gibco, Grand Island, NY, United States), rinsed in sterile PBS in a culture hood and trimmed. Eight cm long segments were cut and tied with suture onto fittings within a bioreactor setup (FIGS. 12A-12C). The vessels were subjected to pulsatile flow as defined by a custom LabVIEW program as previously described (Atigh et al., 2017). The flow medium was made up of Dulbecco’s modified eagle’s medium containing 10% fetal bovine serum and 1% antibiotic-antimycotic. Prior to deployment, vessel diameter was evaluated by ultrasound and vessels were denuded with balloon angioplasty. The drug coated balloon angioplasty were deployed for 2 min at a 10–20% overstretch. At 1 hour, flow was ceased and the treated portion of the vessel was removed. Excised vessels were flash frozen, stored at –80°C and shipped on dry ice to iC42 Clinical Research and Development (Aurora, CO, United States) for quantification of arterial paclitaxel using a validated high-performance liquid chromatography (HPLC)-electrospray ionization- tandem mass spectrometry system (LC-MS/MS).

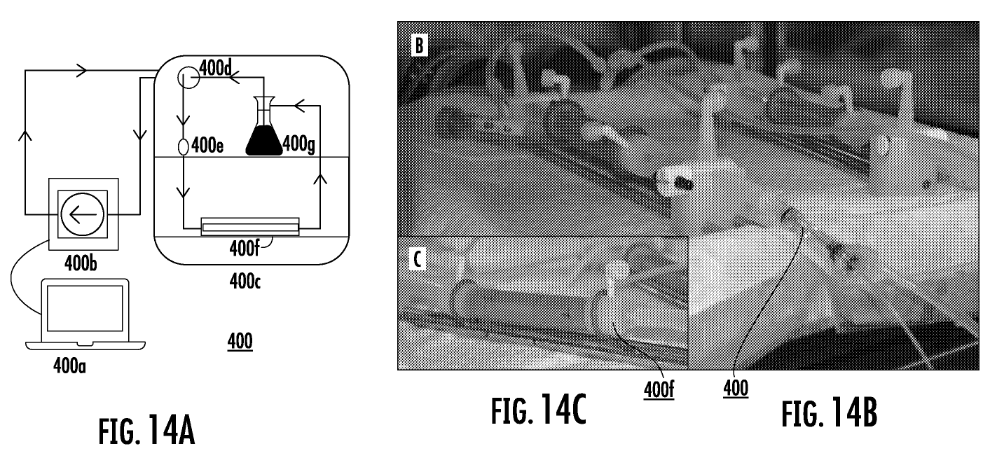

Referring to FIG. 14A, a schematic of the ex vivo flow circuit 400 is shown. A computer 400a controls a pump 400b that is capable of generating pulsatile flow conditions. The tubing from the gear pump 400b passes through a port 400d of the CO2 incubator 400c. The culture medium then passes through a low-pass filter 400e and through the lumen of the explanted arteries in the bioreactor housing compartment 400f and into the flow reservoir 400g. FIGS. 14B and 14C are images of the bioreactor housing compartment 400f. The coated balloons were able to be delivered using the catheter based-devices to the explanted pig artery. Due to the optical clarity of the system, the treatment site where the balloons are inflated within can be clearly viewed and identified in the artery.

### Results

### Drug seeding of silicone elastic tubes

Silicone elastic tubing with an inner diameter of 6 mm was successfully created. Following the seeding process, liquid paclitaxel, dissolved paclitaxel and dissolved sirolimus were observed to have been coated on the luminal surface of the tubes as observed by SEM imaging.

### Transfer of drug from silicone tubing to uncoated balloon angioplasty

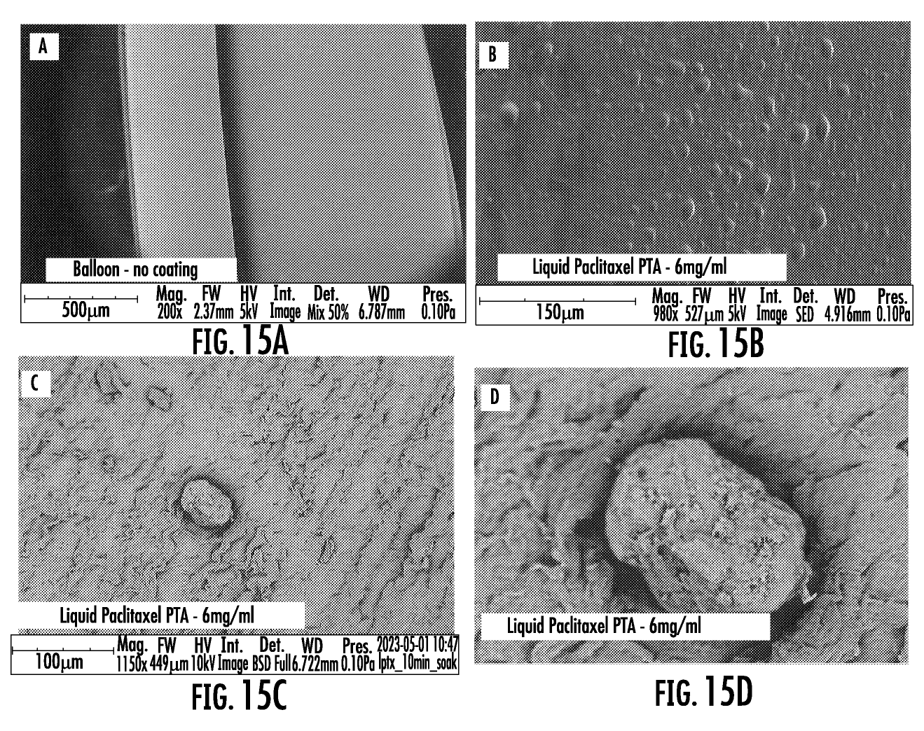

Following the deployment of the uncoated balloon within the silicone tubing, drug was observed to be transferred to the balloon surface (FIG. 15B). SEM analysis was able to show the presence of drug (liquid paclitaxel, dissolved paclitaxel and dissolved sirolimus) on the surface of the balloon angioplasty.

### Scanning Electron Microscopy Visualization of Drug Transfer

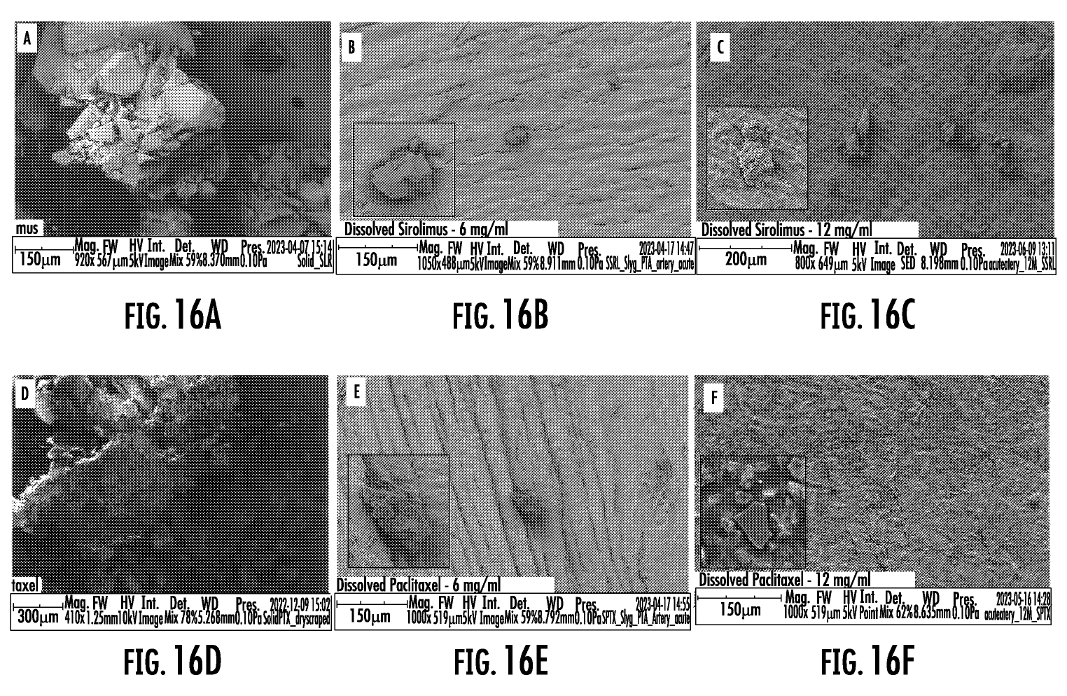

SEM images of the treated arteries displayed the transfer of drug from the balloon surface to the arterial wall. These included liquid sirolimus (FIGS. 15C, 15D) and dissolved sirolimus (FIGS. 16A, 16B, 16C) and dissolved paclitaxel (FIGS. 16E, 16F). Furthermore, increase in drug concentration showed higher drug deposits on the surface for the dissolved sirolimus and paclitaxel.

FIG. 16A is an SEM image of dissolved sirolimus showing phenotype characteristics. FIGS. 16B and 16C are SEM images of the luminal surface of an artery showing drug transfer from a balloon coated with dissolved sirolimus at 6 mg/mL (FIG. 16B) and 12 mg/mL (FIG. 16C). FIG. 16D is an SEM image of dissolved paclitaxel showing phenotype characteristics. FIGS. 16E and 16F are SEM images of the luminal surface of an artery showing drug transfer from a balloon coated with dissolved paclitaxel at 6 mg/mL (FIG. 16E) and 12 mg/mL (FIG. 16F).

### Arterial tissue pharmacokinetics

The 1-hour time point was chosen to investigate the acute transfer and retention of drug within vessel tissue. The arteries treated with uncoated balloon angioplasty coated with liquid paclitaxel (6 mg/mL) retained 4.3 ± 2.68 ng/mg at 1-hour post-delivery.

### Discussions

The pharmacokinetic results using the ex vivo model determined if the arterial paclitaxel levels were within the therapeutic target ranging from 0.01 to 100 ng/mg (paclitaxel amount/weight of arterial tissue)²⁵. The ex vivo results demonstrated the treated tissue had arterial paclitaxel levels of 4.3 ± 2.68 ng/mg at 1-hour post-delivery. And although these studies will need to be repeated in vivo, the utilized bench-top model has been validated using parallel animal models for both drug coated balloons and drug eluting stents²⁶.

In this study, the capability to coat the uncoated balloons with either paclitaxel or sirolimus (anti-proliferative) was demonstrated. However, it is worth noting any therapeutic drug targeting proliferation, migration or inflammation can be coated onto the surface. Additionally, the method can be incorporated and modified so that the plain old balloon angioplasty can be dipped into any solution. In this study, contrast agent was used primarily because this is readily available in the clinic and it has shown to be an effective drug carrier/excipient for anti-proliferative drugs^{27,28}. However other solutions can be utilized or added within the process and provided at the surgical site.

In a first in vivo animal testing, a balloon angioplasty balloon was coated with paclitaxel coating by deploying the balloon within the silicone tubing coated by 6 mg/ml of dissolved solid paclitaxel with contrast as the excipient. The coated balloon was then deployed within the external iliac artery of a pig. At 1 hour post-delivery, the tissue drug levels were at 1.61 ± 1.19 ng/mg.

In earlier work, at 1 hour post-delivery, the tissue drug levels were at 4.3 ± 2.68 ng/mg using the ex vivo model. This was accomplished by coating the silicone tubing by 6 mg/ml of liquid paclitaxel.

In new experiments, the tissue drug levels were increased to 8.65 ± 5.39 ng/mg quantified using the ex vivo model. This was accomplished by coating the silicone tubing by 12 mg/ml of dissolved solid paclitaxel with contrast as the excipient.

FIG. 18 is an SEM image of 12 mg/ml of dissolved solid paclitaxel spray coated onto silicone tubing using the spray coating system shown in FIG. 17A.

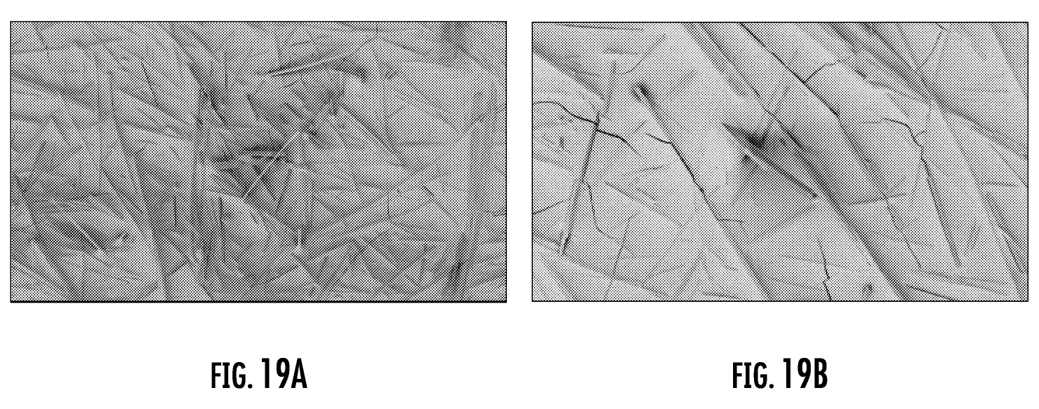

FIGS. 19A and 19B are SEM images showing a coating on an inner surface of a silicone tube using 12 mg/ml of dissolved solid, FDA-grade, paclitaxel. with contrast as the excipient. The paclitaxel was dissolved in ethanol and mixed with the excipient and then used to coat the inner of the silicone tubing.

The contrast agent was selected as it is an FDA approved product. However, it is contemplated that other excipient can be used. For the FDA-grade paclitaxel, the drug seems to adhere to the silicone tubing better than other drugs and there was no need to pre-coat it with a contrast agent (for the tubing). To transfer to the balloon, a pre-coating with contrast may be preferred, although when used in real practice, it may not be needed given the balloon will likely have already been exposed to circulating blood and the proteins associated with any artery that may have been opened by the balloon.

### Conclusions

The present disclosure provides a new approach to coat uncoated angioplasty balloons with therapeutic drug using a drug-lined tube. The drug has been successfully coated on the inner surface of an elastic tube and transferred to an uncoated angioplasty balloon. Pharmacokinetic analysis demonstrated successful transfer and retention of the drug from the drug-coated angioplasty balloon. The first in vivo animal study supports the feasibility of this approach. The approach provides a more cost-effective alternative to the high upfront costs of currently available commercial DCBs which has prevented its use in millions of patients.

The idea of local drug delivery technology has been used in coronary and lower limb interventions since the 1990s.³⁵ The concept is to combine conventional angioplasty with local drug release at the affected area, thus preventing neointimal hyperplasia and recurrence of stenosis. The most commonly used drug in drug-eluting balloons is paclitaxel, which is a cytotoxic agent with hydrophobic-lipophilic properties that facilitate cellular uptake and deliverability of the drug.³⁶ Once paclitaxel is released, it stops the cell cycle in the M-phase of mitosis, preventing neointimal hyperplasia by causing cellular apoptosis and inhibition of vascular smooth muscle cell migration into the intima.³⁷ In 2017, the FDA approved the first drug-coated balloon (DCB) to treat stenosis in a dialysis vascular access. The method of drug delivery is critical, as very slow or very fast delivery could result in subtherapeutic levels, and the desired outcome may not be seen. The use of a DCB is a promising therapy, with recent studies suggesting better outcomes in the short term but inconclusive long term durable results.

In the drawings and specification, there have been disclosed embodiments of the invention and, although specific terms are employed, they are used in a generic and descriptive sense only and not for purposes of limitation, the scope of the invention being set forth in the following claims.

Thus, the foregoing is illustrative of the present invention and is not to be construed as limiting thereof. More particularly, the workflow steps may be carried out in a different manner, in a different order and/or with other workflow steps or may omit some or replace some workflow steps with other steps. Although a few exemplary embodiments of this invention have been described, those skilled in the art will readily appreciate that many modifications are possible in the exemplary embodiments without materially departing from the novel teachings and advantages of this invention.

Accordingly, all such modifications are intended to be included within the scope of this invention as defined in the claims. In the claims, means-plus-function clauses, where used, are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

References (all of which are hereby incorporated by reference as if recited in full herein).

### References

1. Norgren L, Hiatt WR, Dormandy JA, Nehler MR, Harris KA, Fowkes FG, Bell K, Caporusso J, Durand-Zaleski I, Komori K, et al. Inter-Society Consensus for the Management of Peripheral Arterial Disease (TASC II). European journal of vascular and endovascular surgery : the official journal of the European Society for Vascular Surgery. 2007;33 Suppl 1:S1-75. doi: 10.1016/j.ejvs.2006.09.024

2. Tsetis D, Belli AM. Guidelines for stenting in infrainguinal arterial disease. Cardiovascular and interventional radiology. 2004;27:198-203. doi: 10.1007/s00270-004-0029-1

3. Schillinger M, Sabeti S, Dick P, Amighi J, Mlekusch W, Schlager O, Loewe C, Cejna M, Lammer J, Minar E. Sustained benefit at 2 years of primary femoropopliteal stenting compared with balloon angioplasty with optional stenting. Circulation. 2007;115:2745-2749. doi: 10.1161/circulationaha.107.688341

4. Tosaka A, Soga Y, Iida O, Ishihara T, Hirano K, Suzuki K, Yokoi H, Nanto S, Nobuyoshi M. Classification and clinical impact of restenosis after femoropopliteal stenting. J Am Coll Cardiol. 2012;59:16-23. doi: 10.1016/j.jacc.2011.09.036

5. Stone GW, Ellis SG, Cox DA, Hermiller J, O'Shaughnessy C, Mann JT, Turco M, Caputo R, Bergin P, Greenberg J, et al. A polymer-based, paclitaxel-eluting stent in patients with coronary artery disease. N Engl J Med. 2004;350:221-231. doi: 10.1056/NEJMoa032441

6. Morice MC, Serruys PW, Sousa JE, Fajadet J, Ban Hayashi E, Perin M, Colombo A, Schuler G, Barragan P, Guagliumi G, et al. A randomized comparison of a sirolimus-eluting stent with a standard stent for coronary revascularization. N Engl J Med. 2002;346:1773-1780. doi: 10.1056/NEJMoa012843

7. Scheinert D, Scheinert S, Sax J, Piorkowski C, Braunlich S, Ulrich M, Biamino G, Schmidt A. Prevalence and clinical impact of stent fractures after femoropopliteal stenting. J Am Coll Cardiol. 2005;45:312-315. doi: 10.1016/j.jacc.2004.11.026

8. Hwang CW, Wu D, Edelman ER. Physiological transport forces govern drug distribution for stent-based delivery. Circulation. 2001;104:600-605.

9. Shishehbor MH, Jaff MR, Beckman JA, Misra S, Schneider PA, Lookstein R, Kashyap VS, Aronow HD, Jones WS, White CJ. Public Health Impact of the Centers for Medicare and Medicaid Services Decision on Pass-Through Add-On Payments for Drug-Coated Balloons: A Call to Action. JACC Cardiovascular interventions. 2018;11:496-499. doi: 10.1016/j.jcin.2018.01.233

10. Marques L, Hopf-Jensen S, Preiss M, Mueller-Huelsbeck S. An Update on Drug-eluting Technology in Peripheral Arteries to Treat Peripheral Arterial Disease. Heart Int. 2021;15:73-78. doi: 10.17925/hi.2021.15.2.73

11. DePietro DM, Trerotola SO. Choosing the right treatment for the right lesion, Part II: a narrative review of drug-coated balloon angioplasty and its evolving role in dialysis access maintenance. Cardiovasc Diagn Ther. 2023;13:233-259. doi: 10.21037/cdt-22-497

12. Zilio F, Verdoia M, De Angelis MC, Zucchelli F, Borghesi M, Rognoni A, Bonmassari R. Drug Coated Balloon in the Treatment of De Novo Coronary Artery Disease: A Narrative Review. J Clin Med. 2023;12. doi: 10.3390/jcm12113662

+13. Montelione N, Catanese V, Nenna A, Jawabra M, Verghi E, Loreni F, Nappi F, Lusini M, Mastroianni C, Jiritano F, et al. The Diagnostic Value of Circulating Biomarkers and Role of Drug-Coated Balloons for In-Stent Restenosis in Patients with Peripheral Arterial Disease. Diagnostics (Basel). 2022;12. doi: 10.3390/diagnostics12092207

14. Shazly T, Torres WM, Secemsky EA, Chitalia VC, Jaffer FA, Kolachalama VB. Understudied factors in drug-coated balloon design and evaluation: A biophysical perspective. Bioeng Transl Med. 2023;8:e10370. doi: 10.1002/btm2.10370

15. Schnorr B, Albrecht T. Drug-coated balloons and their place in treating peripheral arterial disease. Expert review of medical devices. 2013;10:105-114. doi: 10.1586/erd.12.67

16. Schorn I, Malinoff H, Anderson S, Lecy C, Wang J, Giorgianni J, Papandreou G. The Lutonix® drug-coated balloon: A novel drug delivery technology for the treatment of vascular disease. Advanced drug delivery reviews. 2017;112:78-87. doi: 10.1016/j.addr.2017.05.015

17. Peterson S, Hasenbank M, Silvestro C, Raina S. IN.PACT Admiral drug-coated balloon: Durable, consistent and safe treatment for femoropopliteal peripheral artery disease. Advanced drug delivery reviews. 2017;112:69-77. doi: 10.1016/j.addr.2016.10.003

18. Rosenfield K, Jaff MR, White CJ, Rocha-Singh K, Mena-Hurtado C, Metzger DC, Brodmann M, Pilger E, Zeller T, Krishnan P, et al. Trial of a Paclitaxel-Coated Balloon for Femoropopliteal Artery Disease. N Engl J Med. 2015;373:145-153. doi: 10.1056/NEJMoa1406235

19. Schneider PA, Laird JR, Tepe G, Brodmann M, Zeller T, Scheinert D, Metzger C, Micari A, Sachar R, Jaff MR, et al. Treatment Effect of Drug-Coated Balloons Is Durable to 3 Years in the Femoropopliteal Arteries: Long-Term Results of the IN.PACT SFA Randomized Trial. Circulation Cardiovascular interventions. 2018;11:e005891. doi: 10.1161/circinterventions.117.005891

20. Lookstein RA, Haruguchi H, Ouriel K, Weinberg I, Lei L, Cihlar S, Holden A. Drug-Coated Balloons for Dysfunctional Dialysis Arteriovenous Fistulas. N Engl J Med. 2020;383:733-742. doi: 10.1056/NEJMoa1914617

21. Holden A, Haruguchi H, Suemitsu K, Isogai N, Ross J, Hull J, Wang H, Seamans J, Lookstein R. IN.PACT AV Access Randomized Trial: 12-Month Clinical Results Demonstrating the Sustained Treatment Effect of Drug-Coated Balloons. J Vasc Interv Radiol. 2022;33:884-894.e887. doi: 10.1016/j.jvir.2022.03.606

22. Trerotola SO, Lawson J, Roy-Chaudhury P, Saad TF. Drug Coated Balloon Angioplasty in Failing AV Fistulas: A Randomized Controlled Trial. Clin J Am Soc Nephrol. 2018;13:1215-1224. doi: 10.2215/cjn.14231217

23. Trerotola SO, Saad TF, Roy-Chaudhury P. The Lutonix AV Randomized Trial of Paclitaxel-Coated Balloons in Arteriovenous Fistula Stenosis: 2-Year Results and Subgroup Analysis. J Vasc Interv Radiol. 2020;31:1-14.e15. doi: 10.1016/j.jvir.2019.08.035

24. Todd M, Nair PK, Ohayon J, Pettigrew RI, Yazdani SK. Liquid Drug Delivery Approaches for the Treatment of Occlusive Arterial Disease: A Systematic Review. J Endovasc Ther. 2022:15266028221120755. doi: 10.1177/15266028221120755

25. Axel DI, Kunert W, Goggelmann C, Oberhoff M, Herdeg C, Kuttner A, Wild DH, Brehm BR, Riessen R, Koveker G, et al. Paclitaxel inhibits arterial smooth muscle cell proliferation and migration in vitro and in vivo using local drug delivery. Circulation. 1997;96:636-645.

26. Cooper K, Cawthon CV, Goel E, Atigh M, Christians U, Yazdani SK. The Development of an ex vivo Flow System to Assess Acute Arterial Drug Retention of Cardiovascular Intravascular Devices. Frontiers in Medical Technology. 2021;3:24. doi: 10.3389/fmedt.2021.675188

27. Scheller B, Speck U, Schmitt A, Bohm M, Nickenig G. Addition of paclitaxel to contrast media prevents restenosis after coronary stent implantation. J Am Coll Cardiol. 2003;42:1415-1420.

28. Scheller B, Speck U, Romeike B, Schmitt A, Sovak M, Bohm M, Stoll HP. Contrast media as carriers for local drug delivery. Successful inhibition of neointimal proliferation in the porcine coronary stent model. European heart journal. 2003;24:1462-1467.

What is claimed is:

1. A system for providing a drug coated balloon of a balloon catheter for a medical procedure, comprising:
a tube configured to couple to an intravascular introducer sheath or provided by a longitudinally extending segment of the intravascular introducer sheath, the tube comprising an inner surface surrounding a lumen, wherein the inner surface comprises at least one therapeutic agent deposited thereon; and
a catheter comprising a balloon coupled to the introducer sheath, wherein the balloon is sized and configured to slidably reside in the lumen of the tube then slidably extended into position in the patient to target anatomy through the intravascular sheath for a medical procedure, and wherein the balloon is expandable in the lumen of the tube to contact the inner surface for the defined period whereby the at least one therapeutic agent is applied to the balloon to thereby provide a drug coated balloon for the medical procedure.

2. The system of claim 1, wherein the balloon is precoated with an excipient that resides between the inner surface and the at least one therapeutic agent.

3. The system of claim 2, wherein the excipient comprises a contrast agent.

4. The system of claim 1, wherein the at least one therapeutic agent inhibits and/or blocks restenosis of an artery, stented artery, vein, stented vein or urethra.

5. The system of claim 1, wherein the tube comprises an elongate body with opposing first and second end portions, and wherein at least one of the first and second end portions comprises a valved luer fitting.

6. The system of claim 1, wherein the tube has an elastic body surrounding the lumen and providing the inner surface.

7. The system of claim 1, wherein the tube has an inelastic and/or substantially rigid body surrounding the lumen and providing the inner surface.

8. The system of claim 1, wherein the tube is provided as a component of a kit for angioplasty balloon therapy.

9. The system of claim 1, wherein prior to use the tube has an inner diameter in a range of 1.5 mm to 15 mm.

10. The system of claim 1, wherein the tube is sufficiently compliant to correspond to compliancy of a blood vessel, and wherein the balloon expands from a deflated configuration to conformably reside against the inner surface of the tube for coating the balloon, then is deflated again and advanced through the intravascular introducer sheath to the target anatomy.

11. The system of claim 1, wherein the tube is coupled to the intravascular sheath at a location that is external to the patient.

12. The system of claim 11, wherein the tube comprises a valve between a connector of the intravascular sheath and a distal end of the tube.

13. The system of claim 1, wherein the tube is provided as a longitudinally extending segment of the intravascular sheath at a location that is distal to a proximal connector of the intravascular sheath.

14. The system of claim 13, further comprising a conduit extending laterally from a proximal end portion of the intravascular sheath in fluid communication with the tube.

15. The system of claim 1, further comprising a guidewire sized and configured to slidably extend through the catheter and the intravascular sheath.

16. A method for providing a drug coated balloon for a medical procedure, comprising:
providing a balloon catheter;
providing a tube with an inner surface comprising at least one therapeutic agent;
providing an intravascular introducer sheath;
coupling the intravascular introducer sheath to the tube; then
inserting a balloon of the balloon catheter into a lumen of the tube and expanding the balloon therein so that an outer surface of the expanded balloon contacts the inner surface of the tube;
transferring at least some of the at least one therapeutic agent onto the outer surface of the expanded balloon to provide a drug coated balloon;
at least partially deflating the drug coated balloon; and
extending the drug coated balloon through the intravascular sheath to target anatomy thereby providing an in situ drug coated balloon for a medical procedure.

17. The method of claim 16, wherein the drug coated balloon is configured to deliver a therapeutic amount of the at least one therapeutic agent to a target intrabody site of a patient.

18. The method of claim 16, wherein the medical procedure is a balloon angioplasty.

19. The method of claim 16, further comprising holding the expanded balloon in the lumen for a defined time, optionally in a range of 30 seconds-10 minutes, for the transfer of at least some of the at least one therapeutic agent.

20. A method for providing a drug coated balloon for a medical procedure, comprising:
providing a balloon catheter;
providing an intravascular introducer sheath comprising a longitudinally extending segment with an inner wall comprising an inner surface comprising at least one therapeutic agent;
coupling the intravascular introducer sheath to the tube; then
inserting a balloon of the balloon catheter into a lumen of the tube and expanding the balloon therein so that an outer surface of the expanded balloon contacts the inner surface of the tube;
transferring at least some of the at least one therapeutic agent onto the outer surface of the expanded balloon to provide a drug coated balloon;
at least partially deflating the drug coated balloon; and
extending the drug coated balloon through the intravascular sheath to target anatomy thereby providing an in situ drug coated balloon for a medical procedure.

21. The method of claim 20, wherein the drug coated balloon is configured to deliver a therapeutic amount of the at least one therapeutic agent to a target intrabody site of a patient.

22. The method of claim 20, wherein the medical procedure is a balloon angioplasty.

23. An intravascular introducer sheath system comprising:
an intravascular introducer sheath comprising a proximal end and an opposing distal end, wherein the intravascular introducer sheath comprises a sheath body coupled at a proximal end to a connector, wherein the sheath body surrounds a longitudinally extending lumen bounded by an inner surface of a wall of the sheath body, and wherein a longitudinally extending segment of the inner surface of the sheath body comprises at least one therapeutic agent configured to coat a balloon of a balloon catheter.

24. The intravascular introducer sheath system of claim 23, wherein the connector is a luer connector sized and configured to slidably receive the balloon of the catheter when the balloon is in a deflated state.

25. The intravascular introducer sheath system of claim 23, wherein the at least one therapeutic agent blocks or inhibits restenosis, optionally wherein the at least one therapeutic agent comprises paclitaxel and/or sirolimus.
